(19) United States
(12) Patent Application Publication
(10) Pub. No.: US 2024/0399352 A1
(43) Pub. Date: Dec. 5, 2024

(54) SECOND GENERATION CATALYSTS FOR REVERSIBLE FORMIC ACID DEHYDROGENATION

(71) Applicant: University of Southern California, Los Angeles, CA (US)

(72) Inventors: Van DO, Los Angeles, CA (US); Valeriy CHEREPAKHIN, Los Angeles, CA (US); Jacob KELBER, Glencoe, IL (US); Travis Jesse WILLIAMS, Los Angeles, CA (US)

(73) Assignee: University of Southern California, Los Angeles, CA (US)

(21) Appl. No.: 18/731,998

(22) Filed: Jun. 3, 2024

Related U.S. Application Data
(60) Provisional application No. 63/470,859, filed on Jun. 3, 2023.

(51) Int. Cl. B01J 31/24; B01J 31/20; B01J 31/22; C07C 51/00; C07F 15/00

(52) U.S. Cl. CPC B01J 31/24; B01J 31/20; B01J 31/223; C07C 51/00; C07F 15/004; B01J 2231/625; B01J 2531/0211; B01J 2531/827

(57) ABSTRACT

The synthesis, structure, and reactivity of a family of iridium-based molecular cluster complexes, including neutron and X-ray single-crystal diffraction measurements, are provided. It is found that one complex, which includes an Ir₃H₆(μ³—H) trinuclear core, exhibits reactivity for reversible CO₂ hydrogenation via hydride transfer. This reactivity is unlike previous reports of one known Ir₃H₆(μ³—H) complex, which is inert.

### CROSS-REFERENCE TO RELATED APPLICATIONS

This application claims the benefit of U.S. provisional application Ser. No. 63/470,859 filed Jun. 3, 2023, the disclosure of which is hereby incorporated in its entirety by reference herein.

### STATEMENT REGARDING FEDERALLY SPONSORED RESEARCH OR DEVELOPMENT

This invention was made with government support under Grant No(s). DE-EE0008825 and DE-EE0011096, awarded by the United States Department of Energy (DOE), and CHE1757942, awarded by the National Science Foundation (NSF). The government has certain rights in the invention.”

### TECHNICAL FIELD

In at least one aspect, the present invention is related to organometallic complexes that catalyze the hydrogenation of carbon dioxide and the decomposition of formic acid.

### BACKGROUND

Metal hydride complexes are ubiquitous in catalysis, [1-3] and polynuclear hydride clusters have unique reactivity due to synergy among multiple metals and ligands. [4] Such cooperation is seen in the activation of small molecules, such as benzene, [5] N₂, [6] CO, [7] and CO₂. [4,8] Developing new catalysts that upcycle CO₂ is especially important for managing atmospheric CO₂. [9] Formate is one desirable product, as it and formic acid have many uses, including H₂ storage. [10] It is also a desirable commodity chemical used as a preservative and antibacterial agent in livestock feed. [11]

Accordingly, there is a need for new catalyst systems for hydrogenating carbon dioxide and for dehydrogenating formic acid.

### SUMMARY

In at least one aspect, a new family of IrₓH_{y} cluster compounds, including a novel Ir₃H₆ (μ³—O) oxo complex, 2-O, and an Ir₃H₆ (μ³—H) complex, 2-H was synthesized. These compounds were then structurally and spectroscopically characterized, and the creativity thoroughly studied their reactivity for the first time. Single crystal X-ray diffraction (SCXRD) and single crystal neutron diffraction (SCND) measurements were to thoroughly structurally characterize the Ir₃H₆ (μ³—H) complex and determine the crystallographic location of the hydride ions.

In another aspect, a catalytic organometallic complex includes a trinuclear iridium complex configured to hydrogenate carbon dioxide (CO₂) and dehydrogenate formic acid. The complex includes iridium coordinated with a ligand comprising a pyridyl group and a phosphine group.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

For a further understanding of the nature, objects, and advantages of the present disclosure, reference should be had to the following detailed description, read in conjunction with the following drawings, wherein like reference numerals denote like elements and wherein:

FIG. 1. Examples of Iridium Complexes.

FIG. 2. Scheme 1. Mechanism of catalytic CO₂ hydrogenation by 2-H via metal-metal cooperation.

FIG. 3. Scheme 2. Generation of trinuclear complexes 2-H, 2-O, and 2-S.

FIG. 4. Scheme 3. Hydrogenation of [1]OTf in methanol and CD₂Cl₂.

FIG. 5. Kinetic profile of [1]OTf hydrogenation derived from ¹H NMR data.

FIG. 6. Molecular structure of the cation in [2-H](OTf)₂×CH₂Cl₂ showing localized hydride ligands (50% probability ellipsoids). Hydrogen atoms of the PN ligands are omitted for clarity.

FIG. 7. Scheme 4 providing oxidation of [2-H](PF₆)₂. Compounds [2-Au](PF₆) (BF₄), [5](PF₆)₃×¼CH₂Cl₂, [8]PF₆, and [10]×CH₂Cl₂ were characterized by X-ray crystallography (FIG. S63).

FIG. 8. Synthesis and characterization of compound [2-H](PF₆)₂.

FIG. 9. Synthesis and characterization of compound [2-H-d₇](PF₆)₂.

FIG. 10. Synthesis and characterization of compound [1-d₂₄]OTf.

FIG. 11. Synthesis and characterization of compound [2-H-d₇₂](PF₆)₂.

FIG. 12. Synthesis and characterization of compound [2-O]OTf (FIG. 13)

FIG. 13. Synthesis and characterization of compound [2-Au](PF₆)₂.

FIG. 14. Synthesis and characterization of compound [5](PF₆)₃.

FIG. 15. Synthesis and characterization of compound [8]PF₆.

FIG. 16. Synthesis and characterization of compound [10]×CH₂Cl₂

FIG. 17. Generation of complexes 2-H, 2-O, and 2-S.

FIG. 18. Reaction between [2-H](PF₆)₂ and AgOAc.

FIG. 19. Reaction between [2-H](PF₆)₂ and Hg(OAc)₂.

FIG. 20. Preparation of compound VII from complex 2-H.

FIG. 21. Mechanistic study showing the formation of organometallic complexes 16 and IVa and IVb.

### DETAILED DESCRIPTION

Reference will now be made in detail to presently preferred compositions, embodiments and methods of the present invention, which constitute the best modes of practicing the invention presently known to the inventors. The Figures are not necessarily to scale. However, it is to be understood that the disclosed embodiments are merely exemplary of the invention that may be embodied in various and alternative forms. Therefore, specific details disclosed herein are not to be interpreted as limiting, but merely as a representative basis for any aspect of the invention and/or as a representative basis for teaching one skilled in the art to variously employ the present invention.

Except in the examples, or where otherwise expressly indicated, all numerical quantities in this description indicating amounts of material or conditions of reaction and/or use are to be understood as modified by the word “about” in describing the broadest scope of the invention. Practice within the numerical limits stated is generally preferred. Also, unless expressly stated to the contrary: all R groups (e.g. Rᵢ where i is an integer) include hydrogen, alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups and M is a metal and L′ is a negative counterion; single letters (e.g., “n” or “o”) are 1, 2, 3, 4, or 5; in the compounds disclosed herein including compounds described by formula or by name, a CH bond can be substituted with alkyl, lower alkyl, C₁₋₆ alkyl, C₆₋₁₀ aryl, C₆₋₁₀ heteroaryl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃ M⁺, —COO⁻M⁺, —CF₂H, —CF₂R′, —CFH₂, and —CFR′R″ where R′, R″ and R′″ are C₁₋₁₀ alkyl or C₆₋₁₈ aryl groups and M is a metal atom (e.g., Na, K, Li, etc.); percent, “parts of,” and ratio values are by weight; the term “polymer” includes “oligomer,” “copolymer,” “terpolymer,” and the like; molecular weights provided for any polymers refers to weight average molecular weight unless otherwise indicated; the description of a group or class of materials as suitable or preferred for a given purpose in connection with the invention implies that mixtures of any two or more of the members of the group or class are equally suitable or preferred; description of constituents in chemical terms refers to the constituents at the time of addition to any combination specified in the description, and does not necessarily preclude chemical interactions among the constituents of a mixture once mixed; the first definition of an acronym or other abbreviation applies to all subsequent uses herein of the same abbreviation and applies mutatis mutandis to normal grammatical variations of the initially defined abbreviation; and, unless expressly stated to the contrary, measurement of a property is determined by the same technique as previously or later referenced for the same property.

The term “alkyl” refers to C₁₋₂₀ inclusive, linear (i.e., “straight-chain”), branched, saturated or at least partially and in some cases fully unsaturated (i.e., alkenyl and alkynyl) hydrocarbon chains, including for example, methyl, ethyl, propyl, isopropyl, butyl, isobutyl, tert-butyl, pentyl, hexyl, octyl, ethenyl, propenyl, butenyl, pentenyl, hexenyl, octenyl, butadienyl, propynyl, butynyl, pentynyl, hexynyl, heptynyl, and allenyl groups. “Branched” refers to an alkyl group in which a lower alkyl group, such as methyl, ethyl or propyl, is attached to a linear alkyl chain. “Lower alkyl” refers to an alkyl group having 1 to about 8 carbon atoms (i.e., a C₁₋₈ alkyl), e.g., 1, 2, 3, 4, 5, 6, 7, or 8 carbon atoms. “Higher alkyl” refers to an alkyl group having about 10 to about 20 carbon atoms, e.g., 10, 11, 12, 13, 14, 15, 16, 17, 18, 19, or 20 carbon atoms.

It is also to be understood that this invention is not limited to the specific embodiments and methods described below, as specific components and/or conditions may, of course, vary. Furthermore, the terminology used herein is used only for the purpose of describing particular embodiments of the present invention and is not intended to be limiting in any way.

It must also be noted that, as used in the specification and the appended claims, the singular form “a,” “an,” and “the” comprise plural referents unless the context clearly indicates otherwise. For example, reference to a component in the singular is intended to comprise a plurality of components.

The term “comprising” is synonymous with “including,” “having,” “containing,” or “characterized by.” These terms are inclusive and open-ended and do not exclude additional, unrecited elements or method steps.

The phrase “consisting of” excludes any element, step, or ingredient not specified in the claim. When this phrase appears in a clause of the body of a claim, rather than immediately following the preamble, it limits only the element set forth in that clause; other elements are not excluded from the claim as a whole.

The phrase “consisting essentially of” limits the scope of a claim to the specified materials or steps, plus those that do not materially affect the basic and novel characteristic(s) of the claimed subject matter.

With respect to the terms “comprising,” “consisting of,” and “consisting essentially of,” where one of these three terms is used herein, the presently disclosed and claimed subject matter can include the use of either of the other two terms.

The phrase “composed of” means “including,” “comprising,” or “having.” Typically, this phrase is used to denote that an object is formed from a material.

It should also be appreciated that integer ranges explicitly include all intervening integers. For example, the integer range 1-10 explicitly includes 1, 2, 3, 4, 5, 6, 7, 8, 9, and 10. Similarly, the range 1 to 100 includes 1, 2, 3, 4 . . . 97, 98, 99, 100. Similarly, when any range is called for, intervening numbers that are increments of the difference between the upper limit and the lower limit divided by 10 can be taken as alternative upper or lower limits. For example, if the range is 1.1. to 2.1 the following numbers 1.2, 1.3, 1.4, 1.5, 1.6, 1.7, 1.8, 1.9, and 2.0 can be selected as lower or upper limits. In the specific examples set forth herein, concentrations, temperature, and reaction conditions (e.g. pressure, pH, etc.) can be practiced with plus or minus 50 percent of the values indicated rounded to three significant figures. In a refinement, concentrations, temperature, and reaction conditions (e.g., pressure, pH, etc.) can be practiced with plus or minus 30 percent of the values indicated rounded to three significant figures of the value provided in the examples. In another refinement, concentrations, temperature, and reaction conditions (e.g., pH, etc.) can be practiced with plus or minus 10 percent of the values indicated rounded to three significant figures of the value provided in the examples.

In the examples set forth herein, concentrations, temperature, and reaction conditions (e.g., pressure, pH, flow rates, etc.) can be practiced with plus or minus 50 percent of the values indicated rounded to or truncated to two significant figures of the value provided in the examples. In a refinement, concentrations, temperature, and reaction conditions (e.g., pressure, pH, flow rates, etc.) can be practiced with plus or minus 30 percent of the values indicated rounded to or truncated to two significant figures of the value provided in the examples. In another refinement, concentrations, temperature, and reaction conditions (e.g., pressure, pH, flow rates, etc.) can be practiced with plus or minus 10 percent of the values indicated rounded to or truncated to two significant figures of the value provided in the examples.

It should be appreciated that positively charged complexes are balanced with a sufficient number of negative counterions to achieve charge neutrality. Examples of negative counterions include but are not limited to PF₆, chloride (Cl⁻), bromide (Br⁻), and iodide (I⁻); pseudohalides like cyanide (CN⁻), thiocyanate (SCN⁻), and azide (N₃⁻); complex anions such as tetrafluoroborate (BF₄⁻), hexafluorophosphate (PF₆⁻), perchlorate (ClO₄⁻), and tetraphenylborate (BPh₄⁻); carboxylates including acetate (CH₃COO⁻), formate (HCOO⁻), and trifluoroacetate (CF₃COO⁻); sulfates and sulfonates such as sulfate (SO₄²⁻), methylsulfonate (CH₃SO₃⁻), and p-toluenesulfonate (TsO⁻ or p-CH₃C₆H₄SO₃⁻); phosphates and phosphonates like dihydrogen phosphate (H₂PO₄⁻) and hydrogen phosphate (HPO₄²⁻); and carbonates including carbonate (CO₃²⁻) and bicarbonate (HCO₃⁻).

Throughout this application, where publications are referenced, the disclosures of these publications in their entirety are hereby incorporated by reference into this application to more fully describe the state of the art to which this invention pertains.

In at least one aspect, a catalytic organometallic complex includes a trinuclear iridium complex configured to hydrogenate carbon dioxide (CO₂) and dehydrogenate formic acid. The complex includes iridium coordinated with a ligand comprising a pyridyl group and a phosphine group.

In another aspect, a catalytic organometallic complex having formula I is provided:

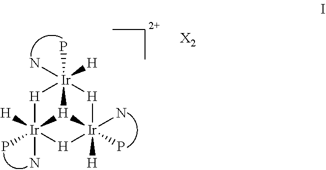

wherein:
X is a counterion having a charge of −1. Alternatively, X₂ can be a bivalent anions such as sulfate (SO₄²⁻), carbonate (CO₃²⁻), and the like; and

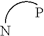

is a bidentate ligand having nitrogen and phosphorus as coordinating atoms. Sometimes this ligand is abbreviated as “NP.”

In another aspect,

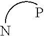

is

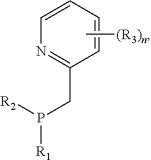

wherein:
n is 0, 1, 2, 3, and 4;
R₁, R₂ are C₁₋₁₀ alkyl; and
R₃ is C₁-10 alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃ M⁺, —COO⁻M⁺, —CF₂H, —CF₂R′, —CFH₂, and —CFR′R″ where R′, R″ and R′″ are C₁₋₁₀ alkyl and M is a metal atom. In a refinement, R₁, R₂ are each independently methyl, ethyl, butyl, n-propyl, isopropyl, n-butyl, sec-butyl, t-butyl. In a refinement, R₁, R₂ are t-butyl. In a refinement, R₃ is C₁-10 alkyl.

In another aspect, a method for hydrogenation CO₂ to form formic acid and/or a formate is provided. The method includes a step of contacting the organometallic complex having formula I or the refinements and variations described above with CO₂ and H₂.

In another embodiment, a method for the dehydrogenation of formic acid is provided. The method includes a step of contacting the organometallic complex having formula I and the refinements and variations described above with formic acid. Typically, the dehydration of formic acid forms hydrogen gas (H₂) and carbon dioxide (CO₂).

In another embodiment, an organometallic complex having formula II is provided:

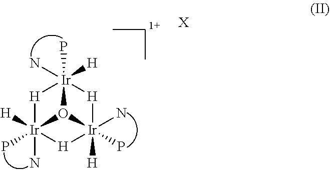

wherein:
X is a counterion having a charge of −1; and

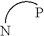

is a bidentate ligand having nitrogen and phosphorus as coordinating atoms.

In a refinement, as set forth above:

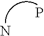

is

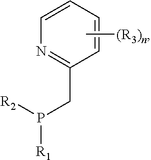

wherein:
n is an integer from 0 to 4;
R₁, R₂ are C₁₋₁₀ alkyl; and
R₃ is C₁-10 alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl and M is a metal atom. In a refinement, R₁, R₂ are each independently methyl, ethyl, butyl, n-propyl, isopropyl, n-butyl, sec-butyl, t-butyl. In a refinement, R₁, R₂ are t-butyl. In a refinement, R₃ is C₁-10 alkyl.

In another embodiment, a method for hydrogenation CO₂ to form formic acid and/or a formate is provided. The method includes a step of contacting the organometallic complex having formula II and the refinements and variations described above with CO₂ and H₂.

In another embodiment, a method for the dehydrogenation of formic acid is provided. The method includes a step of contacting the organometallic complex having formula II and the refinements and variations described above with formic acid. Typically, the dehydration of formic acid forms hydrogen gas (H₂) and carbon dioxide (CO₂).

In another embodiment, an organometallic complex having formula III is provided:

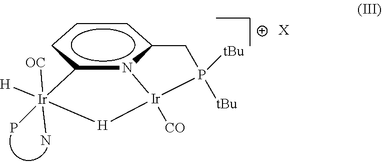

wherein:
X is a counterion having a charge of −1; and

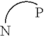

is a bidentate ligand having nitrogen and phosphorus as coordinating atoms.

In a refinement, as set forth above:

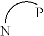

is

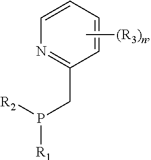

n is an integer from 0 to 4;
R₁, R₂ are C₁₋₁₀ alkyl; and
R₃ is C₁-10 alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ 16 9, wherein R₁, R₂ are each independently methyl, ethyl, butyl, n-propyl, isopropyl, n-butyl, sec-butyl, t-butyl.

In another embodiment, a method for hydrogenation of CO₂ to form a formate includes a step of contacting the organometallic complex having formula II and the refinements and variations described above with CO₂ and H₂.

In another embodiment, a method for dehydrogenation of formic acid includes a step of contacting the organometallic complex having formula III or the refinements and variations of the organometallic complex having formula 3 described above with formic acid.

In another embodiment, an organometallic complex having formula IVA or IVB is provided:

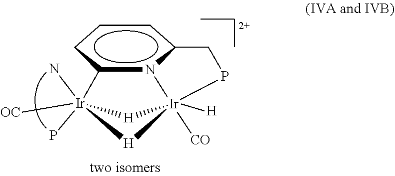

wherein:
X is a counterion having a charge of −1; and

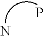

is a bidentate ligand having nitrogen and phosphorus as coordinating atoms. The structure of IV is stereogenic at both of the iridium centers. NMR shows that there are two diastereomers present in solution, both of which conform to the structural diagram shown.

In a refinement, as set forth above,

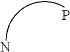

is

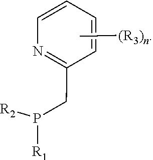

n is an integer from 0 to 4;
R₁, R₂ are C₁₋₁₀ alkyl; and
R₃ is C₁-10 alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃ M⁺, —COO⁻M⁺, —CF₂H, —CF₂R′, —CFH₂, and —CFR′R″ where R′, R″ and R′″ are C₁₋₁₀ 16 9, wherein R₁, R₂ are each independently methyl, ethyl, butyl, n-propyl, isopropyl, n-butyl, sec-butyl, t-butyl.

In another embodiment, a method for hydrogenation of CO₂ to form a formate includes a step of contacting the organometallic complex having formula IVA or IVB or the refinements and variations of the organometallic complex having formula IVA or IVB with CO₂ and H₂.

In another aspect, a method for dehydrogenating formic acid includes contacting the organometallic complex having formula IVA or IVB or the refinements and variations of the organometallic complex having formula IVA or IVB with formic acid.

In another embodiment, a method comprising reacting an organic metal compound having formula I and the variations set forth above with carbon monoxide in a solvent to form an organometallic complex having formula V:

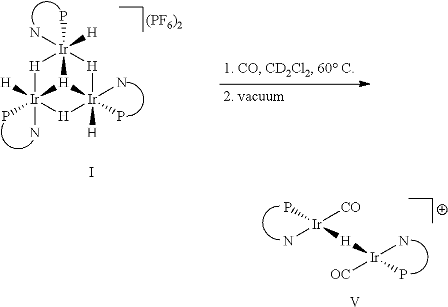

Details for the organometallic complex 1 are set forth above.

In another embodiment, a method comprising reacting an organ metal compound having formula 1 or the refinements and variations of the organometallic complex having formula I with formic acid in a solvent to form an organometallic complex having formula VI:

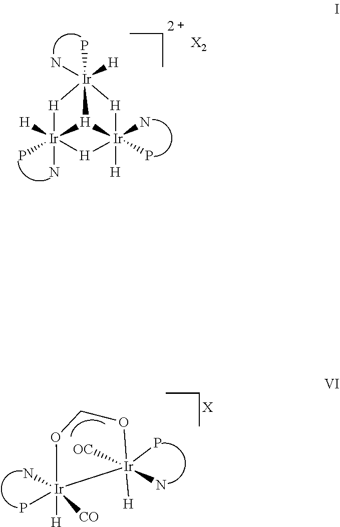

wherein X is a counterion having a charge of −1 or X₂ is a bivalent anion. For example, the reaction can proceed as follows:

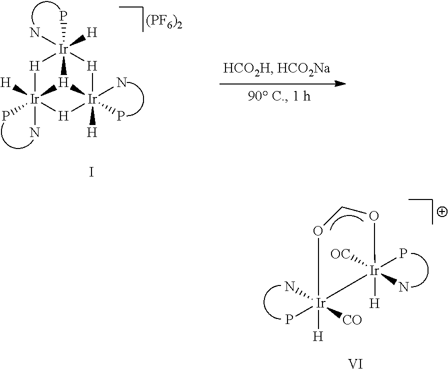

Details for the organometallic complex I are set forth above.

In another aspect, an organometallic complex having formula VII is provided:

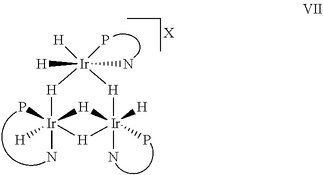

wherein X is a counterion having a charge of −1. This complex can be made by treating complex I with hydrogen gas in the presence of a base (e.g., K₂CO₃), which results in opening the cluster at its μ₃ hydride with the uptake of H₂. This results in the installation of an eighth hydride in the cluster with concurrent reduction in charge from +2 to +1.

In another aspect, a catalytic system for the conversion of CO₂ to formate and vice versa is provided. The catalyst system includes a catalytic organometallic complex having formula I or II. The catalytic system also includes an aqueous solution that includes a base such as potassium carbonate (K₂CO₃). The conditions of temperature and pressure suitable for the reversible conversion between CO₂ and formate. In a refinement, the system achieves a combined turnover number for both hydrogenation and dehydrogenation greater than 10,000.

In another aspect, a process for preparing the catalytic compound of formula I is provided. The method includes a step of reacting an iridium precursor with a ligand containing (2-pyridyl) CH₂PBut₂ under conditions suitable for forming the triiridium heptahydride complex [Ir₃H₆ (μ³—H)(PN)₃]²⁺. In a refinement, the iridium precursor is selected from the group consisting of IrCl₃, IrBr₃, and IrI₃. In a refinement, the method further includes a step of isolating the catalytic compound in a crystalline form suitable for neutron crystallography.

In another aspect, a catalytic kit is provided. The catalytic kit includes an organometallic complex having formula I, II, II, and optionally, instructions for use in CO₂ hydrogenation or formic acid dehydrogenation. In a refinement, the catalytic kit further includes one or more of the following: a solvent, a co-catalyst, and a container for mixing the reaction components.

In another aspect, suitable reaction conditions for the hydrogenation reactions include a temperature in the range of 50 to 120° C. and a pressure in the range of 35 to 90 bar. In a refinement, the ratio of H₂ to CO₂ is maintained at an appropriate level such as 4:1 to 1:1. Reaction times can be from about 1 hour to 70 hours. Suitable solvents include but are not limited to, water. For example, an aqueous solution of a base, such as potassium carbonate, optionally with tetrahydrofuran (THF), can be used. In a refinement, the concentration of the organometallic complex in the hydrogenation reactions is from about 20 ppm to about 3000 ppm.

In another aspect, suitable reaction conditions for the dehydrogenation reactions include a temperature in the range of 80 to 120° C. and a pressure in the range of 1 to 90 bar. Suitable solvents include but are not limited to, water. In a refinement, a co-catalyst such as sodium formate (HCOONa) can be used. In a refinement, the co-catalyst can be percent in an amount from about 1 mole percent to about 100 mole percent of the amount of formic acid. In a refinement, the concentration of the organometallic complex in the dehydrogenation reactions is from about 20 ppm to about 3000 ppm.

The following examples illustrate the various embodiments of the present invention. Those skilled in the art will recognize many variations that are within the spirit of the present invention and scope of the claims.

### 1.1 Introduction

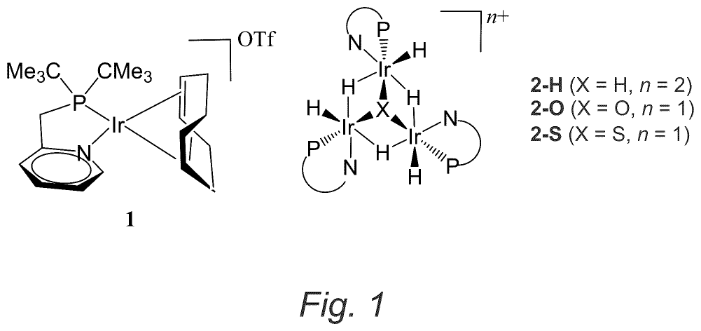

The special catalytic activity from multinuclear polyhydride clusters IrₓH_{y} in the hydrogenation of CO₂ to formate is investigated. While hundreds of mononuclear and dinuclear iridium hydride species are known, [8e, 12] there are only five reports on triiridium heptahydride complexes [Ir₃H₆ (μ³—H) L₆]²⁺. The first is a deactivated form of Crabtree's catalyst in his olefin hydrogenation system: Crabtree (1978), [13] Pignolet (1980 and 1988), Albinati (2003), [15] and Inagaki (2018) [16] all point out the catalytic inertness and high stability of this scaffold. Yet, it is hypothesized that in the right ligand environment, Ir₃H₇ systems may have attractive catalytic properties. In the experiments set forth below, the chemistry of this complex is developed, optimized as 2-H (FIG. 1), as a reversible CO₂ hydrogenation catalyst. Moreover, the mystery of how an apparently inert cluster can be made to become a useful tool for global carbon management is probed.

### 1.2 Results and Discussion

### 1.2.1 Catalytic Hydrogenation of CO₂

Compound [2-H](PF₆)₂ does not hydrogenate organic substrates such as 1-hexene (acetone-d₆, 60° C.) or acetophenone (neat, 90° C.), which is consistent with the previously-reported inactivity of [Ir₃H₇L₆]²⁺ clusters. [13b, 14,15] Nevertheless, it was found that 2-H is catalytically active in CO₂ hydrogenation and formic acid dehydrogenation (Eq. 1, 2). The catalytic activity of [1]OTf, [2-O]OTf, and [2-H](PF₆)₂ were tested in hydrogenating aqueous solutions of K₂CO₃ under 35-90 bar of H₂/CO₂ at 50-120° C. (Table 1). Complex 2-O is the least active (TON=141) among the three catalysts at 50° C., while 1 and 2-H show the same activity (entries 1-3). There is no effect of pressure on the performance of 2-H (entries 3 and 4); however, raising the temperature from 50 to 120° C. improves its TON from 459 to 7,190 (entries 4-6). To evaluate the maximum TON of 2-H, the reaction was run for 24, 48, and 70 hours at 120° C. (entries 6-8) and extrapolate the ultimate deactivation of 2-H after about 4 days at 120° C. with a maximum TON of ca. 10 k cycles per iridium atom or 30 k cycles of the cluster (Table 1, entry 8).

CO₂+K₂CO₃+2H₂→2HCOOK+H₂O (1)

HCOOH→H₂+CO₂ (2)

TABLE 1
Catalytic hydrogenation of CO₂ to HCOOK.[a]

| | Catalyst | P (bar) | K2CO3 | Temp. (° C.) | Yield (%)[c] |
|---|---|---|---|---|---|
| Entry | (ppm)[b] | (H₂:CO₂) | (g) | Time (h) | TON[d] |
| 1 | [1]OTf | 35 (6:1) | 1.00 | 50 | 4 |
| | (101) | | | 24 | 398 |
| 2 | [2-O]OTf | 45 (7:2) | 1.00 | 50 | 2 |
| | (142) | | | 24 | 141 |
| 3 | [2-H](PF₆)₂ | 45 (7:2) | 1.00 | 50 | 5 |
| | (131) | | | 24 | 382 |
| 4 | [2-H](PF₆)₂ | 90 (7:2) | 1.00 | 50 | 6 |
| | (131) | | | 24 | 459 |
| 5 | [2-H](PF₆)₂ | 45 (7:2) | 0.10 | 70 | 100 |
| | (1,307) | | | 24 | 765 |
| 6 | [2-H](PF₆)₂ | 90 (7:2) | 1.00 | 120 | 94 |
| | (131) | | | 24 | 7,190 |
| 7 | [2-H](PF₆)₂ | 88 (7:2) | 5.00 | 120 | 22 |
| | (26) | | | 48 | 8,414 |
| 8 | [2-H](PF₆)₂ | 88 (1:1) | 5.00 | 120 | 25 |
| | (26) | | | 70 | 9,561 |

[a]Conditions: catalyst (1.0 mg in 1.0 mL THF), K₂CO₃, H₂O (5 mL), H₂/CO₂.
[b]ppm(Ir) = [mol Ir at.]/[mol K⁺] × 10⁶.
[c]Yields were derived from ¹H NMR spectra using DMF as an internal standard. Yield % = 6 × [int. HCO₂]/[int. HCONMe₂] × [mol DMF]/[mol K⁺] × 100%.
[d]TON = [mol K⁺]/[mol Ir at.] × Yield %/100%. All TONs are with respect to one Ir atom.

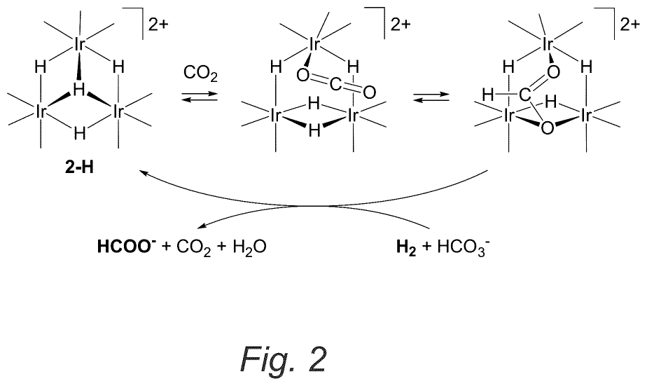

Like metal-ligand cooperation, metal-metal cooperation can also enable CO₂ hydrogenation, since activation of CO₂ and stabilization of HCOO⁻ product could involve coordination to multiple centers in the catalytic ion. [4] Bicarbonate is not basic enough to activate the PN ligand by CH₂ deprotonation, so the metal-metal cooperation seems a more plausible option to access a cooperative bond activation mechanism. One such mechanism is illustrated in Scheme 1 (FIG. 2).

The hypothesis of Scheme 1 (FIG. 2) was interrogated with a ¹H NMR experiment in which CO₂ was hydrogenated at room temperature and ambient pressure (reagents: [2-H](PF₆)₂, H₂/CO₂ (1:1), K₂CO₃, H₂O, and CD3OD). After 1 day, free formate (δH=8.55 ppm) and partial derivatization of 2-H were detected, evinced by hydride peaks between −18 and −30 ppm. Recharging the system with 1 bar of H₂/CO₂ increased the amount of formate without changing the ratio between 2-H and it's in situ derivatives. This is consistent with a dynamic equilibrium among catalytic species and 2-H as a resting state. Unfortunately, the very small quantities of the catalytic intermediates impeded their structural characterization.

Dehydrogenation of neat formic acid was performed using [2-H](PF₆)₂ and HCOONa co-catalyst at reflux (110° C., oil bath). Once fully dehydrogenated, a solid catalytic mixture was recharged with another portion of formic acid. Thus 33 mL of formic acid in 11 cycles over the course of 4 days were converted, which corresponds to at least 54,400 turnovers per Ir atom (>163 k TON per cluster). NMR studies reveal clean conversion of complex 2-H to its post-catalytic forms in both formic acid dehydrogenation and CO₂ hydrogenation at high temperatures and pressures.

Inspired by the discovery of this unique reactivity, which is completely unprecedented among analogous iridium clusters, the uniqueness of 2-H and the proposal of Scheme 1 (FIG. 2) were probed through a system of stoichiometric, crystallographic, and spectroscopic studies.

### 1.2.2 Trinuclear Complexes [Ir₃H₆ (μ³—X) (PN)₃]ⁿ⁺

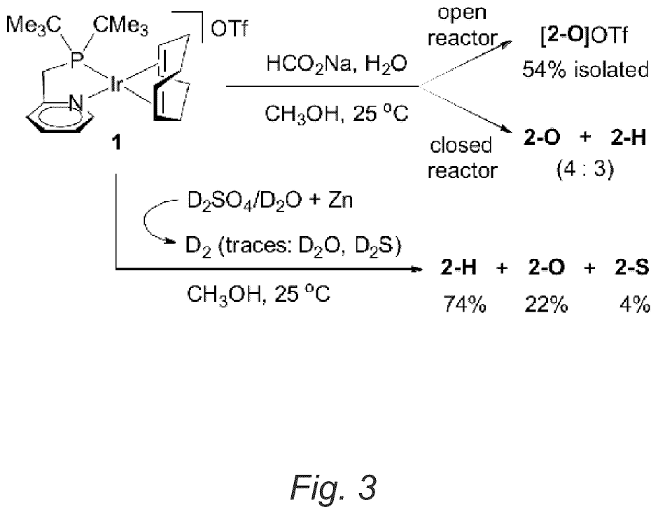

Formation of Trinuclear Complexes. Oxo-centered triiridium cluster [Ir₃H₆ (μ³—O) (PN)₃]⁺ (2-O) were first encountered in experiments on methanol dehydrogenation using [1] OTf as a catalyst (Scheme 2, FIG. 3). Compound [2-O] OTf is obtained in 54% yield by reacting [1] OTf with sodium formate in aqueous methanol. The [2-O]⁺ cation features three equivalent Ir^{III} (PN) fragments bound by a triply-bridging pyramidal oxygen atom characterized by Ir—O distance of 2.071 Å and Ir—O—Ir angles of 86.31° (X-ray). ¹H NMR shows two signals at −22.03 (3H) and −22.49 ppm (3H), corresponding to bridging and terminal hydrides. While there are several complexes with Ir₃ (μ₃—O) core, 2-O is the first example of cluster [Ir₃H₆ (μ₃—O) L₆]⁺.

Isotopic labeling experiments establish the synthetic origin of each hydride in 2-0 (Table 2). Deuteration of the CH₃ group in methanol has no effect on the yield or deuteration of 2—O (entries 3 and 4): methanol dehydrogenation is not a step in its formation. Conversely, dehydrogenation of formate is kinetically-relevant, as evinced by a strong kinetic isotope effect at its CH site (entries 1 and 3). However, deuterium incorporated in 2-O does not originate from formate (entry 2): it derives from H+/D+ exchange with O-deuterated solvent, as exemplified in entries 3 and 4. This unusual observation supports a view that H/D exchange mechanistically precedes cluster formation because equal deuteration of the terminal and bridging hydrides in 2-0 were observed. Surprisingly, the deuterium content in the Ir₃H₆ fragment does not exceed 39%, even when no extra ¹H is added to the system (entry 1). This is interpreted to indicate participation of cyclooctadiene-h₁₂ and CH₂ group of the PN ligand in 1 as ¹H donors.

TABLE 2
Isotopic labeling of 2-0.^{[a]}

| | | NMR yield | Deuteration of |
|---|---|---|---|
| Entry | Reagents | of 2-O (%)^{[b]} | Ir3H₆ (%)^{[c]} |
| | DCO₂Na, CD₃OD, D₂O | 4 | 39 |
| | DCO₂Na, CH₃OH, H₂O | 28 | 0 |
| | HCO₂Na, CD₃OD, D₂O | 51 | 30 |
| | HCO₂Na, CH₃OD, D₂O | 47 | 39 |
| | HCO₂Na, CD₃OH, H₂O | 64 | 0 |

^{[a]}Conditions: [1]OTf (20 mg), Na-formate (20 mg), methanol (0.3 mL), and water (0.3 mL) were stirred under N₂ for 3 h at 25° C. Solvent removal was followed by CD₂Cl₂ (0.7 mL) addition and ¹H NMR analysis.
^{[b]}Yield % = 4 × [int. 9.9 ppm]/[int. total ArH] × 100%.
^{[c]}D % = ([int. 9.9 ppm] − [int. −22.0 ppm])/[int.9.9 ppm] × 100%.

Clean conversion of 1 to 2-O was observed only when the reactor was open to an N₂ atmosphere, whereas in a closed system, the reaction gives 2-O and a reduced tri iridium cluster, 2-H, in a 4:3 ratio (Scheme 2, FIG. 3). Complex 2-H ([Ir₃H₆ (μ₃—H)(PN)₃]²⁺) was identified by the characteristic quadruplet of the triply-bridging hydride ligand (δH=−4.10 ppm, ²J_{trans-PH}=45 Hz). Generally, the syntheses of complexes [Ir₃H₇L₆]²⁺ from Ir(I) precursors require H₂ gas. [13-16] Therefore, it was suspected that 2-H formation involves formate dehydrogenation that accumulates H₂ in the closed system.

Treatment of [1] OTf with D₂ gas, generated from D₂SO₄/D₂O and metallic zinc gives a mixture of trinuclear complexes: 2-H (74%), 2-O (22%), and 2-S (4%). The products show no sign of deuteration suggesting H/D scrambling between D₂ and methanol's OH. The oxo and thio derivatives form due to contamination of D₂ with D₂O and D₂S, respectively. Although [1] OTf readily reacts with H₂/H₂S or HCO₂Na/H₂S in methanol, the reactions do not favor generation of 2-S. (Complex 2-S was characterized in CD₂C₁₂ solution by NMR (δ_{H}=−22.63 and −24.65 ppm, δ_{P}=67.29 ppm) and MALDI-MS (m/z=1326.65 Da).

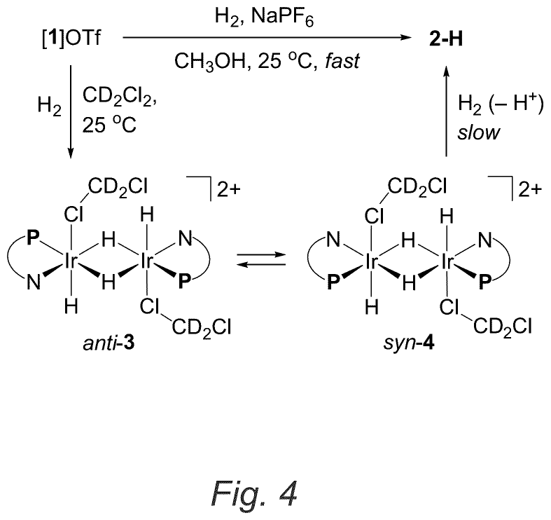

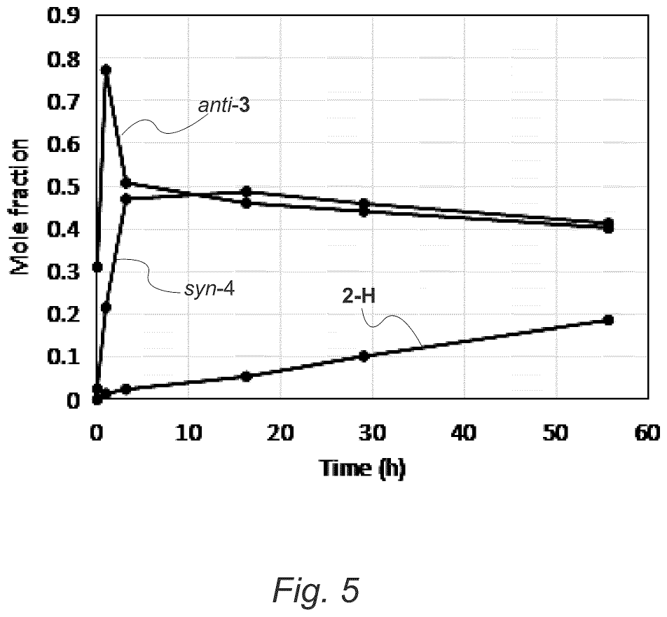

Compounds [2-H](PF₆)₂, [2-H](OTf)₂, [2-H-d₇](PF₆)₂, and [2-H-d₇₂](PF₆)₂ (Scheme 3, FIG. 4) were prepared. [13-16] While 2-H forms quickly in methanol (ca. 10 min), the reaction slows in an aprotic CH₂Cl₂ environment affording evidence of isomeric dinuclear intermediates anti-3 and syn-4 with the composition [Ir₂H₂ (μ-H)₂ (CD₂Cl₂)₂ (PN)₂]²⁺. [17] A time course ¹H NMR study (FIG. 5) shows rapid conversion of 1 to anti-3, with concurrent full hydrogenation of cyclooctadiene, within 1 hour. A dynamic equilibrium between anti-3 and syn-4 (1:1) is then established within 10 hours, which does not change as the reaction continues. Finally, a slow, zero-order conversion of the isomers gives 2-H in 25% NMR yield after 56 hours.

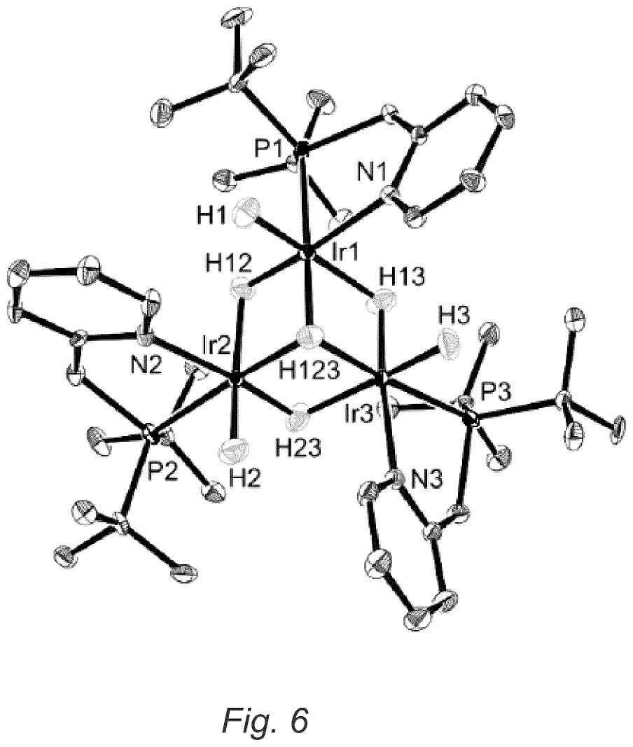

1.2.3 Neutron Crystallography of 2-H. The first single-crystal neutron diffraction study on a triiridium heptahydride complex, particularly [2-H](OTf)₂×CH₂Cl₂ is reported herein. The molecular structure of 2-H is shown in FIG. 6 and its selected interatomic distances are given in Table 3. Complex 2-H features three Ir(III) atoms arranged around a C₃ axis. The iridium atoms adopt a distorted octahedral geometry with the bond angles ranging from 80° to 105°. The composition of 2-H suggests the absence of Ir—Ir bonds; however, the average distance between Ir atoms (2.724 Å) is very close to the length of covalent Ir—Ir bond found in Ir₄ (CO) 12 (2.683 Å). [18]

TABLE 3
Selected interatomic distances (Å) in 2-H
determined by neutron crystallography.

| | | Ir—(μ-H) | Ir—(μ-H) | Ir—(μ-H) | |
|---|---|---|---|---|---|
| | Ir—H | short | long | long | Ir . . . Ir |
| Ir1 | 1.577(7) | 1.682(6) | 1.947(7) | 1.917(7) | Ir3 2.712(3) |
| Ir2 | 1.581(7) | 1.690(7) | 1.949(7) | 1.945(7) | Ir1 2.734(3) |
| Ir3 | 1.581(6) | 1.681(6) | 1.939(7) | 1.925(7) | Ir2 2.727(3) |

Consistent with ¹H NMR data, crystallography reveals the location and coordination modes of seven hydride ligands: three terminal (H1, H2, H3), three bridging (H12, H13, H23), and one triply-bridging hydride (H123). The pyramidal coordination geometry of μ³—H is characterized by an 89.89° angle Ir—(μ³—H)—Ir and 173.73° angle P—Ir—(μ³—H). As expected, average lengths of Ir—H bonds involving terminal H (1.580 Å) and μ³—H (1.929 Å) are consistent with the proposed bond orders. Surprisingly, μ-H groups are asymmetrical, with short (1.684 Å) and long (1.945 Å) Ir-(μ-H) bonds that mimic the lengths of Ir—H and Ir—(μ³—H), respectively, appropriate for a trans effect of the terminal hydrides. While both Ir—(μ³—H) and long Ir-(μ-H) bonds appear structurally lengthened, it is observed that only the Ir—(μ³—H) bond is dissociated during carbonylation of the analogous [Ir₃H7 (dppp) 3]²⁺ to give [Ir₃H3 (μ-H) 4 (CO) (dppp) 3]²⁺. [14b]

### 1.2.4 Stoichiometric Reactivity of 2-H

Electrophilic Substitution of Hydrides. While it reacts with CO₂, complex 2-H is inert to CH₃I, CH₃OTf, and [CPh₃]BF₄, suggesting low nucleophilicity of its hydride ligands. Furthermore, compound [2-H](OTf)₂ demonstrates very surprising stability in neat CF₃SO₃D at 25° C.: ¹H NMR shows no decomposition or HD gas formation. However, the hydride ligands undergo H/D exchange at all three sites with different relative rates. After 3 days at 25° C., it was noticed that the terminal hydrides exchange significantly faster (94% D, δ_{H}=−23.04 ppm) than μ-H (49% D, δ_{H}=−18.56 ppm) and μ³—H (50% D, δ_{H}=−4.10 ppm). No H/D exchange was detected in CD₃OD. This startling was found to be startling. Therefore, synthetic studies of reactions of 2-H with mildly oxidative reagents that could illustrate the reactivity of the respective hydride ligands were pursued which challenges the view of cluster opening advanced in Scheme 1 (FIG. 2).

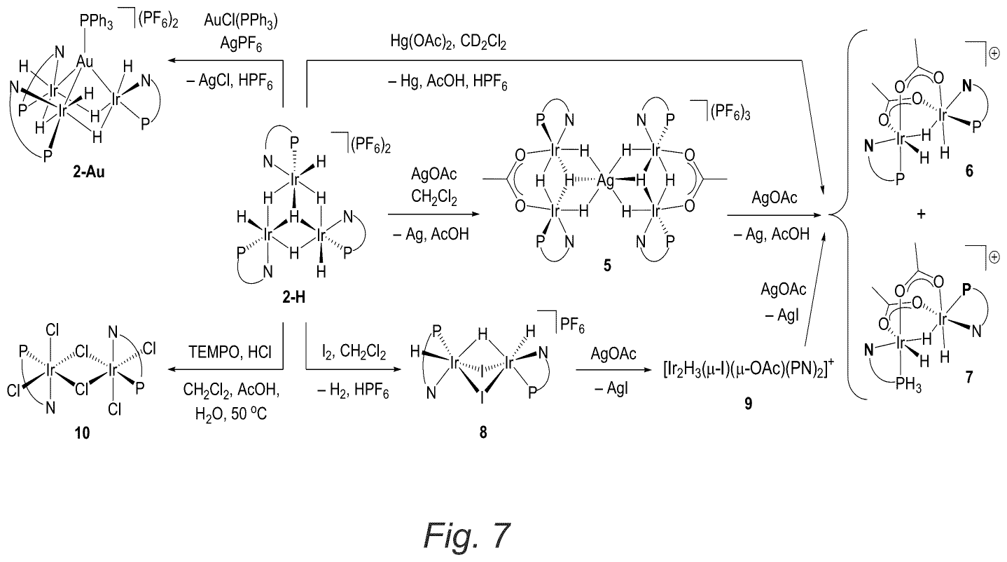

It has been shown that the central μ³—H can be displaced selectively by various electrophiles. For example, treating [2-H](PF₆)₂ with AuPPh₃⁺, generated in situ from AuCl(PPh₃) and AgPF₆, gives tetranuclear complex [Ir₃H₆ (μ³—AuPPh₃) (PN)₃]²⁺ (2-Au, Scheme 4, FIG. 7). In contrast to 2-H and 2-O, 2-Au adopts nearly perfect tetrahedral geometry with Ir—Ir and Ir—Au distances ranging between 2.74 Å and 2.81 Å. The Au—P bond is tilted by 13° from the expected principal C3 axis. ³¹P NMR, however, shows that 2-Au is perfectly symmetric in a CD₂Cl₂ solution. While the average distance Au—Ir found in 2-Au (2.779 Å) is consistent with those in structures featuring R₃PAu (η³—Ir₃) and Ir₃H₆ (μ³—AuX) cores: [Ph₃PAu {Ir₆Ru(CO)₁₅}]⁻ (2.838 Å), [22]Ph₃PAu {Ir₆Ru₃ (CO)₂₁}]⁻ (2.831 Å), and [Ir₃H₆ (μ³—AuNO₃)(dppe)₃]⁺ (2.705 Å), it is not clear whether Au and Ir are bound directly or via bridging hydrides. Strong spin coupling between the phosphorus nuclei of PN and P Ph₃ (³J_{PP}=41 Hz) in ³¹P NMR favors three covalent bonds Au—Ir. This description is also consistent with ²J_{PH} coupling constants of the hydride ligands (δ_{H}=−20.61 and −20.97 ppm) that were not affected by the installation of AuPPh₃ group. The auration of 2-H can be viewed either as electrophilic substitution or as gold-induced intramolecular oxidation of μ³—H to H+ (Eq. 3).

Ir₃^{III}H₇²⁺+Au⁺→Ir^{III}Ir₂^{II}Au^{I}H₆²⁺+H⁺ (3)

Oxidation of Hydrides. Complex 2-H is stable to aerobic oxidation: [2-H](PF₆)₂ withstands heating to 200° C. in air in a glass capillary. Redox potentials of [2-H](PF₆)₂ were studied by cyclic voltammetry in a solution of CH₂Cl₂/CH₃OH/H₂O. Upon scanning anodically, two chemically irreversible oxidation events were detected at 0.95 V and 1.96 V (vs. SHE at 200 mV/s). Oxidation at 0.95 V seems to cause the removal of μ³—H ligand followed by dissociation of the trinuclear structure. This pathway was interrogated by controlled chemical oxidation using AgOAc, Hg(OAc)₂, and I₂ (Scheme 4, FIG. 7).

Compound [2-H](PF₆)₂ slowly reacts with an excess of AgOAc (E°_{Ag+/Ag}=0.799 V vs. SHE) [25] in CH₂Cl₂ over days, forming a bright orange pentanuclear heterometallic complex [AgIr₄H₈ (μ—OAc)₂(PN)₄]³⁺ (5), that was isolated in 50% yield. It features two identical fragments syn-Ir₂H₄ (μ—OAc)(PN)₂⁺ coordinating Ag⁺ in a centrosymmetric fashion. ¹H NMR data are consistent with eight hydrides respectively at −4.75 (2H), −21.85 (4H), and −30.78 ppm (2H). Average interatomic distances Ir—Ir and Ir—Ag are 2.648 Å and 2.879 Å, respectively, consistent with bonding between the metals via hydride bridges rather than Ag—Ir bonds: Ag—Ir distances in 5 are 0.17 Å longer than in the complex [Ag{(μ-H) 3Ir(PPh₃) 3} 2]⁺ (2.709 Å), also containing AgH₆ polyhedron. [26] The independent synthesis and characterization of complexes anti-[Ir₂H₄ (μ—O₂CR) (PN)₂]⁺ has been documented. [27] Formation of 5 is a window into how 2-H opens. It is envisaged that each metal of 2-H brings two hydrides to 5, leaving one hydride, apparent μ³—H, cleaved oxidatively.

As the reaction with AgOAc continues, complex 5 is oxidized further to a mixture of diastereomeric cations 6 and 7 (1:12 ratio). Complex 6 was previously characterized as a meso isomer of [Ir₂H₃ (μ—OAc)₂ (PN)₂]⁺, thus presaging a route by which 2-H could be converted to a known formic acid dehydrogenation catalyst. Complex 7 is an asymmetric isomer of [Ir₂H₃ (μ—OAc)₂ (PN)₂]⁺ (vide infra).

In contrast to AgOAc, oxidation of [2-H](PF₆)₂ with Hg(OAc)₂ (E°_{Hg2+/Hg}=0.852 V vs. SHE) [25] in CD₂Cl₂ is fast and unselective, as indicated by immediate precipitation of metallic mercury and generation of a complex mixture of products in the resulting solution. However, after 3 days the initially formed intermediates converge to complexes 6 and 7 in a 1:1 ratio. It is inferred that this pathway is likely analogous to the one available for reactions of AgOAc.

Compound [2-H](PF₆)₂ reacts quantitatively with 1.5 equivalents of 12 (E°I₂/I₃-=0.789 V vs. SHE) [25] in CH₂Cl₂ to give a bright yellow dinuclear complex [Ir₂H₃ (μ-I)₂ (PN)₂]⁺ (8). Similar to the Hg(OAc)₂ reaction, the initial fast iodination produces a mixture of intermediates that equilibrate selectively to 8 within 5 hours. H₂ gas was detected as one of the reaction products (δ_{H}=4.60 ppm), which is expected, as further oxidation of H₂ to HI is endergonic (ΔG=+1.7 kJ/mol). Compound [8]PF₆; was isolated in 77% yield. The structure of 8 was confirmed by X-ray crystallography. Unfortunately, the poor quality of its crystals limits us to presenting this structure for connectivity only: all anions could not be localized in the unit cell. The cation contains two distinct Ir(PN) fragments that cause the bridging hydride (δ_{H}=−18.72 ppm) to appear as a doublet of doublets with a larger coupling constant associated with trans-PH spin coupling (²J_{PH}=48 Hz). The same coupling pattern was observed for the bridging hydride in complex 7 (δ_{H}=−19.57 ppm, ²J_{trans-PH}=51 Hz), which supports the structure assignment. Further, treating [8]PF₆ with AgOAc in CD₂Cl₂ solution generates a mixture of acetato derivatives 6 and 7 (3:4 ratio) via asymmetric mixed-ligand intermediate [Ir₂H₃ (μ-I) (μ—OAc)(PN)₂]⁺ (9), which also features the characteristic signal of the bridging hydride (δ_{H}=−18.30 ppm, ²J_{trans-PH}=50 Hz).

The described experiments illustrate high thermodynamic stability of class [Ir₂H₃ (μ-X)₂ (PN)₂]⁺ (X=I, OAc) with selective reactivity of μ³—H ahead of other hydrides in the starting cluster. Still, the remaining hydrides can also be oxidized all together. Under more forcing conditions, TEMPO and aqueous HCl oxidize [2-H](PF₆)₂ to red [Ir₂Cl₄ (μ-Cl)₂ (PN)₂] (10) within 17 hours at 50° C. (Scheme 4, FIG. 7). The product is insoluble in common solvents, but X-ray diffraction-quality crystals were obtained by running the oxidation in AcOH/CH₂Cl₂ solvent system. The distance between Ir atoms in complex 10 (3.715 Å) is ca. 1 Å longer than in its hydride-bridged congeners.

Overall, it was observed that under appropriate oxidative conditions 2-H can generate at least four classes of iridium complexes of varied nuclearity and hydride content: [Ir₃H₆X(PN)₃]²⁺ (X=AuPPh₃), Ir₂H₄X(PN)₃⁺ (X=OAc), [Ir₂H₃X₂ (PN)₂]⁺ (X=I, OAc), and [Ir₂X₆ (PN)₂] (X=Cl). Having screened a wide range of reagents, a correlation between the nature of an oxidant and the reaction kinetics was noticed. While elemental iodine, AuPPh₃⁺, and acetates of Ag(I) and Hg(II) work well at room temperature, due to their ability to coordinate with the hydride ligands or enable direct electron transfer from Ir—H bonds, reagents that lack these properties (NBS, CBr₄, Me₃NO, TEMPO, PhI(OAc)₂, and PhHgOAc) show no reactivity or require forcing conditions (TEMPO/HCl system).

### 1.3 Conclusion

The experiments set forth above regarding complex 2-H involved complete structural characterization. The stability limits of 2-H to oxidation was explored and the hydride source in its synthesis was identified. Complex 2-H demonstrated unexpected catalytic activity in reversible CO₂ hydrogenation, which could be enabled by the metal-metal cooperation within the triiridium cluster. These combined structural and reactivity data support a mechanism in which the Ir₃H₇ cluster initially opened by cleavage of its central μ³ hydride. It is proposed that this is an entry path for CO₂ to bind the cluster and enables CO₂ reduction by transfer of a neighboring hydride. It is expected that this hydride transfer is enabled by opening of the elongated side of one of the μ² hydrides. This is a structural feature of 2-H that is not known for other members of the class and could be responsible for its novel reactivity.

### 1.4 Materials and Methods

Compounds [1] OTf [1a] and [1-d₂₄] OTf were prepared following the same procedure described here. Ligand PN-d₂₄ was synthesized starting from non-deuterated 2-methylpyridine and tert-butanol. [1] Solvents CD₂Cl₂ and CD₃OD were purchased from Cambridge Isotope Laboratories and used without purification. Hexane, dichloromethane, ether, and tetrahydrofuran were dried using solvent purification system. All other reagents were purchased from commercial sources and used without purification.

All manipulations with metal complexes were performed under nitrogen in a Vacuum Atmospheres glovebox (0-10 ppm O₂). High-pressure CO₂ hydrogenation reactions were conducted in a non-stirred 125 mL Parr reactor. ¹H, ²H, ¹³C, ¹⁹F, and ³¹P NMR spectra were recorded on Varian Mercury 400, VNMRS-500, and VNMRS-600 spectrometers and processed using MestReNova 12.0.1. All chemical shifts are reported in ppm and referenced to solvent peaks (¹H, ²H, ¹³C), external 85% H₃PO₄ or internal PF₆⁻ (³¹P). The following abbreviations are used: (s) singlet, (bs s) broad singlet, (d) doublet, (t) triplet, (dd) doublet of doublets, etc. NMR spectra of all metal complexes were acquired in 8″ J. Young tubes (Wilmad or Norell) with Teflon valve plugs. MALDI-MS spectra were acquired on Bruker Autoflex Speed MALDI Mass Spectrometer. Elemental analyses were conducted on Flash 2000 CHNS Elemental Analyzer. Infrared spectra were recorded on Bruker OPUS FTIR spectrometer. Electronic absorption spectra were acquired on Perkin-Elmer UV-vis-NIR spectrometer.

### 1.4.1 Synthesis and Characterization

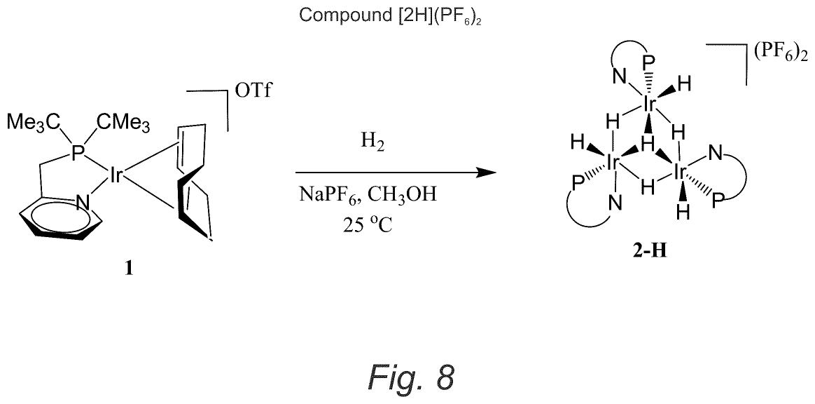

### 1.4.1.1 Compound [2-H](PF₆)₂ (FIG. 8)

3[Ir(C₈H₁₂)(PN)]⁺+10H₂=[Ir₃H₇(PN)₃]²⁺+3C₈H₁₆+H⁺

A suspension of [1] OTf (0.200 g, 2.911×10-4 mol) and NaPF₆ (0.100 g, 5.954×10⁻⁴ mol, 2 eq.) in dry methanol (5 mL) was stirred under one atmosphere of hydrogen gas at 25° C. Within ten minutes [1] OTf completely dissolved, the resulting orange-red solution turned bright yellow, and [2-H](PF₆)₂ partially crystallized. After six hours ether (40 mL) was added and the mixture was left overnight to enable full precipitation of the product. Yellow solid was filtered, washed with ether, and redissolved in CH₂Cl₂. The resulting yellow solution was filtered from salts. Bright yellow crystals of [2-H](PF₆)₂ were obtained by adding ether in small portions to the CH₂Cl₂ solution. The crystals were separated by decantation, washed with ether, and dried in vacuum. Yield: 135 mg (88%).

¹H NMR (600 MHz, CD₂Cl₂): δ 9.33 (d, J=5.7 Hz, 3H, ArH), 7.85 (t, J=7.7 Hz, 3H, ArH), 7.69 (d, J=7.8 Hz, 3H, ArH), 7.29 (t, J=6.6 Hz, 3H, ArH), 3.65 (dd, J=17.6, 8.4 Hz, 3H, 3CH₂), 3.52 (dd, J=17.6, 9.9 Hz, 3H, 3CH₂), 1.32 (d, J=14.0 Hz, 27H, 9CH₃), 1.07 (d, J=14.4 Hz, 27H, 9CH₃), −4.10 (q, ²J_{PH}=45.1 Hz, 1H, μ³—IrH), −18.56 (d, J=7.7 Hz, 3H, IrH), −23.04 (d, J=22.7 Hz, 3H, IrH). ¹³C {¹H} NMR (151 MHZ, CD₂Cl₂): δ 162.81, 158.49, 139.20, 124.13, 123.54, 37.59 (d, ¹J_{PC}=32.8 Hz), 37.02 (d, ¹J_{PC}=28.4 Hz), 35.09 (d, ¹J_{PC}=28.0 Hz), 28.97, 28.51. ¹⁹F {¹H} NMR (564 MHz, CD₂Cl₂): δ-72.97 (d, ¹J_{PF}=711.1 Hz, PF₆). ³¹P {¹H} NMR (243 MHz, CD₂Cl₂): δ 75.34 (s, PN), −145.00 (hept, ¹J_{PF}=711.1 Hz, PF₆). IR (KBr, cm⁻¹): 2969, 2910, 2876, 2257 ν(IrH), 1770 ν(IrH), 1609, 1479, 1448, 1397, 1376, 1187, 1165, 1024, 848 ν (PF₆), 769, 630, 559. ESI-MS: m/z calcd for [C₄₂H₇₉Ir₃N₃P₃]²⁺647.7, found 647.6. Anal. calcd. for C₄₂H₇₉F₁₂Ir₃N₃P₅: C, 31.81; H, 5.02; N, 2.65. Found C, 32.38; H, 4.67; N, 2.84. UV-Vis (CH₃OH): λₘₐₓ=327 nm (ε=12123 M⁻¹ cm⁻¹).

### 1.4.1.2 Compound [2-H](OTf)₂

Method 1: A suspension of [1] OTf (0.200 g, 2.911×10⁻⁴ mol) and NaPF₆ (0.100 g, 5.954× 10⁻⁴ mol, 2 eq.) in dry methanol (5 mL) was stirred under one atmosphere of hydrogen gas at 25° C. Within ten minutes [1] OTf completely dissolved, the resulting orange-red solution turned bright yellow, and [2-H](PF₆)₂ partially crystallized. After 17 hours the solvent was removed in vacuum, and [2-H](OTf)₂ was extracted from the residue with CH₂Cl₂. The extract was filtered from salts, then methanol (1 mL) and excess of ether were added to crystallize bright yellow [2-H](OTf)₂ (131 mg). The material was filtered, redissolved in CH₂Cl₂, and the resulting suspension was again filtered from salts. The solution was evaporated to dryness, and the residue was finally recrystallized from methanol (1 mL) and excess of ether. Yield: 124 mg (80%). In both crystallizations methanol is required to prevent deposition of an oil.

Method 2: A solution of [1] OTf (0.250 g, 3.639×10⁻⁴ mol) in dry methanol (10 mL) was stirred for 1 hour under one atmosphere of hydrogen gas at 25° C. The resulting bright yellow solution was evaporated in vacuum, then the residue was redissolved in CH₂Cl₂ (3 mL), and the solution was layered with ether. Bright yellow crystals of [2-H](OTf)₂×CH₂Cl₂ suitable for the single-crystal neutron diffraction study were formed after several days. Crystals of [2-H](OTf)₂×CH₂Cl₂ appropriate for X-ray analysis were grown from CH₂Cl₂/ether solution.

¹H NMR spectra of [2-H](PF₆)₂ and [2-H](OTf)₂ are identical. ¹⁹F {¹H} NMR (564 MHz, CD₂Cl₂): δ-78.86 (s, CF₃). ³¹P {¹H} NMR (243 MHZ, CD₂Cl₂): δ 76.37 (s, PN). IR (KBr, cm⁻¹): 2962, 2909, 2878, 2256 ν(IrH), 1763 ν(IrH), 1610, 1481, 1446, 1394, 1374, 1272 vas (SO₃), 1227 vs (CF₃), 1156 Vas (CF₃), 1033 vs (SO₃), 827, 779, 763, 641 δₛ (SO₃). Anal. calcd. for C₄₄H₇₉F₆Ir₃N₃O₆P₃S₂: C, 33.16; H, 5.00; N, 2.64. Found C, 33.79; H, 5.09; N, 2.28.

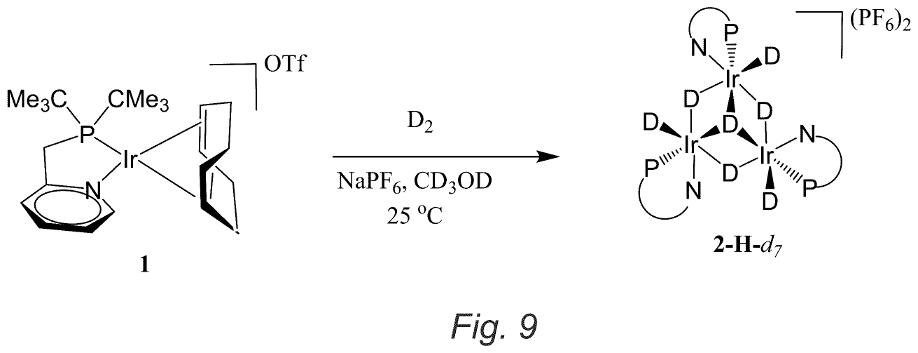

### 1.4.1.3 Compound [2-H-d₇](PF₆)₂ (FIG. 9)

Compound [2-H-d₇](PF₆)₂ was obtained in 87% yield (67 mg) by following the procedure for the synthesis of [2-H](PF₆)₂ and using [1] OTf (100 mg), NaPF₆ (50 mg), methanol-d₄ (2 mL), and D₂ gas.

¹H NMR (600 MHZ, CD₂Cl₂): δ 9.33 (d, J=5.6 Hz, 1H, ArH), 7.85 (t, J=7.7 Hz, 1H, ArH), 7.70 (d, J=7.9 Hz, 1H, ArH), 7.29 (t, J=6.7 Hz, 1H, ArH), 3.65 (dd, J=17.6, 8.5 Hz, 1H, CH₂), 3.53 (dd, J=17.8, 9.8 Hz, 1H, CH₂), 1.33 (d, J=13.8 Hz, 9H, 3CH₃), 1.07 (d, J=14.1 Hz, 9H, 3CH₃). ²H NMR (92 MHZ, CH₂Cl₂): δ-4.07 (q, ²J_{PD})=6.5 Hz, IrD), −18.48 (br s, IrD), −22.81 (br s, IrD). ¹⁹F {¹H} NMR (564 MHz, CD₂Cl₂): δ-72.96 (d, ¹J_{PF}=711.0 Hz, PF₆). ³¹P {¹H} NMR (243 MHz, CD₂Cl₂): δ 75.54 (three-line pattern, ²J_{PD})=6.2 Hz, PN), −145.00 (hept, ¹J_{PF}=711.0 Hz, PF₆). IR (KBr, cm⁻¹): 2966, 2910, 2877, 1609, 1480, 1445, 1393, 1373, 1274, 1256, 1184, 843 ν (PF₆), 762, 559. MALDI-MS: m/z calcd for [C28H₄₈D3Ir₂N₂P₂]⁺864.29, found 864.04. Anal. calcd. for C₄₂H₇₂D7F₁₂Ir₃N₃P₅: C, 31.67; H, 5.44; N, 2.64. Found C, 31.60; H, 4.52; N, 2.69.

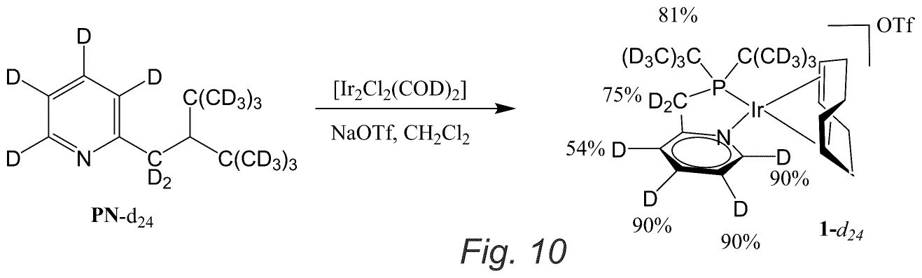

### 1.4.1.4 Compound [1-d₂₄] OTf (FIG. 10)

A solution of ligand PN-d₂₄ (2.96 g, 11.320 mmol) in CH₂Cl₂ (10 mL) was added dropwise to a stirred suspension of [Ir₂Cl₂ (COD)₂] (3.80 g, 5.660 mmol) and CF₃SO₃Na (3.90 g, 22.640 mmol) in CH₂Cl₂ (50 mL). The reaction was stirred for one hour, then the mixture was filtered and concentrated in vacuum. Addition of hexane causes crystallization of ruby-red product [1-d₂₄] OTf, which was filtered, washed with hexane, and dried in vacuum. Yield: 6.36 g (79%).

¹H NMR (600 MHZ, CD₂Cl₂): δ 8.27 (s, ArH, 90% D), 8.11-8.07 (m, ArH, 90% D), 8.03 (s, ArH, 54% D), 7.48 (s, ArH, 90% D), 4.92-4.83 (m, 2H, CH₂), 4.52-4.43 (m, 2H, CH₂), 3.69-3.58 (m, CH₂, 75% D), 2.44-2.31 (m, 2H, CH₂), 2.31-2.12 (m, 4H, 2CH₂), 2.01-1.86 (m, 2H, CH₂), 1.34-1.22 (m, 6CH₃, 81% D). ²H NMR (92 MHZ, CH₂Cl₂): δ 8.30 (br s), 8.14 (br s), 8.08 (br s), 7.53 (br s), 3.64 (br s), 1.29 (br s). ¹³C {¹H} NMR (126 MHZ, CD₂Cl₂): δ 167.31-167.12 (m, Py), 149.66-148.49 (m, Py), 141.79-140.93 (m, Py), 125.09 (d, J=9.0 Hz, Py), 124.46-123.85 (m, Py), 121.29 (q, ¹J_{CF}=321.4 Hz, CF₃), 90.41 (d, J=11.0 Hz, CH), 63.13 (s, CH), 37.15-36.35 (m), 34.57-33.65 (m), 33.37 (td, J=7.6, 2.7 Hz, CH₂), 30.02-28.66 (m, tBu), 28.23 (td, J=4.8, 1.6 Hz, CH₂). ¹⁹F {¹H} NMR (564 MHz, CD₂Cl₂): δ-78.84 (s, CF₃). ³¹P {¹H} NMR (243 MHZ, CD₂Cl₂): δ 58.04-57.10 (m, PN). IR (KBr, cm⁻¹): 2957, 2930, 2895, 2843, 2297, 2231, 2216, 2133, 2069, 1589, 1541, 1433, 1277 νₐₛ (SO₃), 1224 νₛ (CF₃), 1155 νₐₛ (CF₃), 1032 νₛ (SO₃), 638 δ (SO₃). MALDI-MS: m/z calcd for [C₂₂H₁₇D₁₉IrNP]⁺557.34, found 557.49. Anal. calcd. for C₂₃H₁₇D₁₉F₃IrNO₃PS: C, 39.13; H+D, 7.85; N, 1.98. Found C, 39.43; H+D, 5.43; N, 2.31. According to ¹H NMR the PN ligand contains in average 80% deuterium among all 24 CH sites, which corresponds to H₅D₁₉ ratio and the total composition of compound [1-d₂₄] OTf as C₂₃H₁₇D₁₉F₃IrNO₃PS.

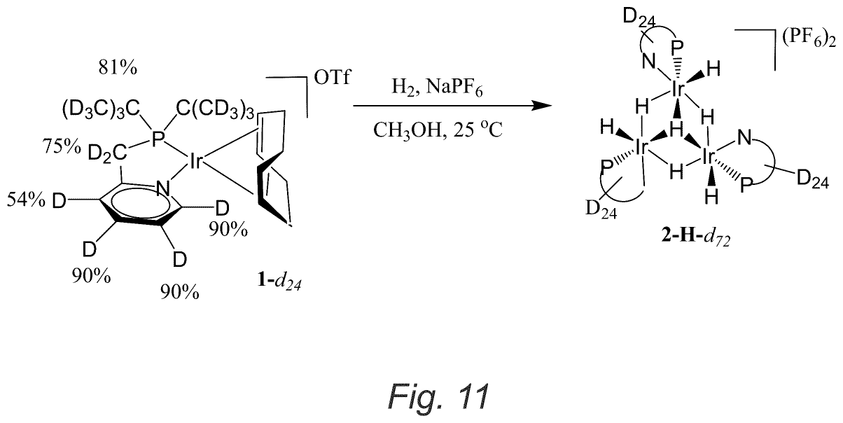

### 1.4.1.5 Compound [2-H-d₇₂](PF₆)₂ (FIG. 11)

The following reagents were transferred to a 1 L Straus flask: [1-d₂₄] OTf (5.00 g, 7.033×10⁻³ mol), NaPF₆ (2.36 g, 1.405×10⁻² mol, 2 eq.), and dry methanol (100 mL). Then, the reactor headspace was filled with hydrogen gas (1 atm) using Schlenk line. The flask was scaled, and the mixture was occasionally shaken to enable dissolution of the reagents and slow crystallization of bright yellow [2-H-d₇₂](PF₆)₂. The reaction was continued for 12 hours during which the reactor was recharged with hydrogen gas three times. The reaction mixture was concentrated in vacuum to ca. 50 mL, then the crude product was filtered, washed with minimum amount of methanol, and dried. Further purification requires dissolution in CH₂Cl₂, filtration from salts, and crystallization by adding ether. Yield: 1.16 g (30%).

¹H NMR (600 MHZ, CD₂Cl₂): δ 9.34 (s, ArH), 7.87-7.82 (m, ArH), 7.70 (s, ArH), 7.29 (s, ArH), 3.70-3.57 (m, CH₂), 3.56-3.46 (m, CH₂), 1.34-1.20 (m, CH₃), 1.09-0.95 (m, CH₃), −4.09 (q, ²J_{PH}=45.2 Hz, 1H, μ³—IrH), −18.58 (s, 3H, 3IrH), −23.03 (d, J=22.13 Hz, 3H, 3IrH). ¹³C {¹H} NMR (151 MHZ, CD₂Cl₂): δ 162.82 (s), 158.12 (m), 138.84 (m), 123.91 (m), 123.52 (s), 36.85 (m), 34.46 (m), 27.98 (m). ¹⁹F {¹H} NMR (564 MHz, CD₂Cl₂): δ-72.96 (d, ¹J_{PF}=711.0 Hz, PF₆). ³¹P {¹H} NMR (243 MHZ, CD₂Cl₂): δ 74.64 (br s, PN), −145.00 (hept, ¹J_{PF}=710.8 Hz, PF₆). IR (KBr, cm⁻¹): 2930, 2233 ν(IrH), 3134, 2075, 1772 ν(IrH), 1590, 1540, 1420, 1384, 1295, 1044, 843 ν (PF₆), 558. MALDI-MS: m/z calcd for [C28H₁₃D₃₈Ir₂N₂P₂]⁺901.52, found 901.44.

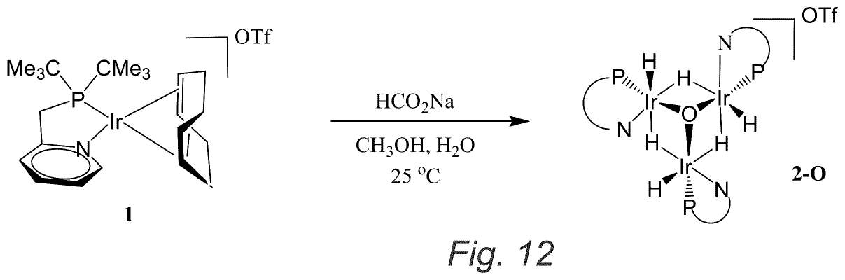

### 1.4.1.6 Compound [2-O] OTf (FIG. 12)

A solution of [1] OTf (0.050 g, 7.278×10⁻⁵ mol) and sodium formate (0.021 g, 3.088×10⁻⁴ mol, 4 eq.) in methanol (0.3 mL) and deionized water (0.3 mL) was stirred in an open reactor at 25° C. for 3 hours. The resulting yellow-orange slurry was evaporated in vacuum to dryness and then the product was extracted with CH₂Cl₂. The solution was filtered from salts, mixed with ether, and left for crystallization in a closed vial. After 2 days an off-white crystalline product [2-O] OTf was formed. It was separated by decantation, washed with ether, and finally dried in vacuum. Yield: 19 mg (54%). Crystals suitable for X-ray analysis were grown from CH₂Cl₂/hexane solution.

¹H NMR (500 MHZ, CD₂Cl₂): δ 9.93 (d, J=5.7 Hz, 1H, ArH), 7.68 (t, J=7.6 Hz, 1H, ArH), 7.47 (d, J=7.8 Hz, 1H, ArH), 7.05 (t, J=6.6 Hz, 1H, ArH), 3.44 (dd, J=17.2, 9.1 Hz, 1H, CH₂), 3.06 (dd, J=17.2, 8.8 Hz, 1H, CH₂), 1.26 (d, ³J_{PH}=13.2 Hz, 9H, 3CH₃), 1.09 (d, ³J_{PH}=13.1 Hz, 9H, 3CH₃), −22.03 (dm, ²J_{PH}=25.0 Hz, 1H, IrH), −22.49 (m, 1H, IrH). ¹³C {¹H}NMR (151 MHz, CD₂Cl₂): δ 165.01, 154.15, 136.57, 122.11, 121.90, 38.14 (d, ¹J_{PC}=26.6 Hz), 34.85 (d, ¹J_{PC}=22.0 Hz), 33.59 (d, ¹J_{PC}=30.1 Hz), 29.64, 29.16. 19F {¹H} NMR (564 MHZ, CD₂Cl₂): δ−78.92 (s, CF₃). ³¹P {¹H} NMR (202 MHZ, CD₂Cl₂): δ 58.08 (dd, J_{PH}=22.6, 5.04 Hz). IR (KBr, cm⁻¹): 2954, 2904, 2873, 2207 ν(IrH), 1738 ν(IrH), 1609, 1480, 1445, 1391, 1370, 1275 νₐₛ (SO₃), 1223 νₛ (CF₃), 1155 νₐₛ (CF₃), 1032 νₛ (SO₃), 823, 770, 638 δₛ (SO₃). MALDI-MS: m/z calcd for [C₄₂H₇₈Ir₃N₃O₄P₃]⁺1310.4, found 1310.1. Anal. calcd. for C₄₃H₇₈F₃Ir₃N₃O₄P₃S: C, 35.38; H, 5.39; N, 2.88. Found C, 34.60; H, 5.20; N, 2.91.

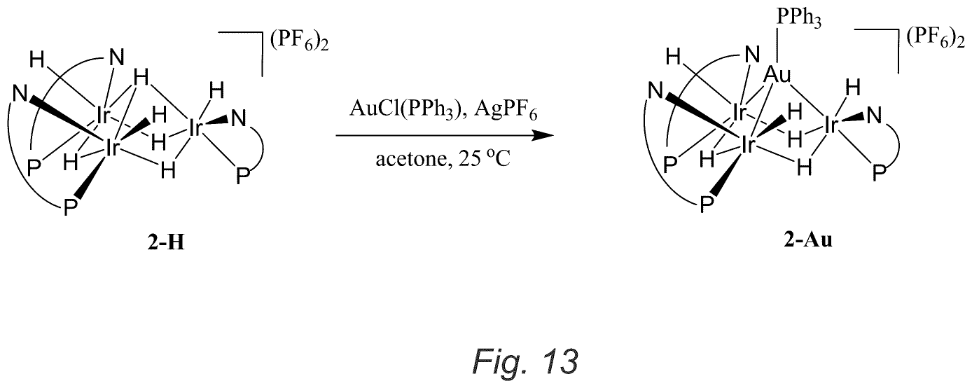

### 1.4.1.7 Compound [2-Au](PF₆)₂ (FIG. 13)

AuCl(PPh₃)+Ag⁺=Au(PPh₃)⁺+AgCl

[Ir₃H₇(PN)₃]²⁺+Au(PPh₃)⁺=[Ir₃H₆(AuPPh₃)(PN)₃]²⁺+H⁺

Silver (I) hexafluorophosphate (0.008 g, 3.164×10⁻⁵ mol) was added to a stirring solution of AuCl(PPh₃) (0.016 g, 3.234×10⁻⁵ mol) in acetone (3 mL) at 25° C. The solution immediately turns cloudy and white AgCl precipitates. The resulting solution was filtered and added dropwise to a stirring solution of [2-H](PF₆)₂ (0.050 g, 3.153×10⁻⁵ mol) in acetone (7 mL). The resulting dark orange mixture was stirred for 5 minutes, then filtered, and evaporated in vacuum to give a dark red glassy solid. The solid was recrystallized from CH₂Cl₂/ether in a closed vial over the course of 2 days yielding bright-red crystals and a black oil. The red crystals of [2-Au](PF₆)₂ were mechanically separated from the oil, washed with ether, and dried in vacuum. Yield: 46 mg (72%). Crystals of [2-Au](PF₆) (BF₄) suitable for X-ray analysis were grown from CH₂Cl₂/ether solution.

¹H NMR (600 MHZ, CD₂Cl₂): δ 10.04 (d, J=5.8 Hz, 3H, ArH), 7.64 (d, J=7.5 Hz, 3H, ArH), 7.56 (t, J=8.0 Hz, 3H, ArH), 7.50 (t, J=7.3 Hz, 3H, ArH), 7.38-7.32 (m, 6H, 3Ph), 7.09-7.03 (m, 6H, 3Ph), 6.30 (t, J=6.5 Hz, 3H, ArH), 3.72 (dd, J=17.3, 8.0 Hz, 3H, 3CH₂), 3.54 (dd, J=17.6, 8.0 Hz, 3H, 3CH₂), 1.24 (d, ³J_{PH}=13.1 Hz, 27H, 9CH₃), 1.18 (d, ³J_{PH}=13.7 Hz, 27H, 9CH₃), −20.61 (d, J=7.0 Hz, 3H, IrH), −20.97 (d, J=21.3 Hz, 3H, IrH). ¹³C {¹H} NMR (151 MHZ, CD₂Cl₂): δ 164.55 (s, Py), 153.89 (s, Py), 138.61 (s, Py), 133.63 (d, J=14.5 Hz, Ph), 132.04 (d, J=2.1 Hz, Ph), 131.02 (d, J=46.4 Hz, Ph), 129.84 (d, J=10.9 Hz, Ph), 123.50 (s, Py), 122.36 (s, Py), 38.64 (d, ¹J_{PC}=27.1 Hz), 37.17 (d, ¹J_{PC}=31.2 Hz), 35.80 (d, ¹J_{PC}=21.9 Hz), 29.47 (s, CH₃), 29.06 (s, CH₃). ¹⁹F {¹H} NMR (564 MHz, CD₂Cl₂): δ-70.58 (d, ¹J_{PF}=710.8 Hz, PF₆). ³¹P {¹H} NMR (243 MHZ, CD₂Cl₂): δ 74.15 (d, ³J_{PP}=40.4 Hz, PN), 59.60 (q, ³J_{PP}=42.3 Hz, PPh₃), −145.00 (hept, ¹J_{PF}=710.8 Hz, PF₆). IR (KBr, cm⁻¹): 2952, 2906, 2877, 2225 ν(IrH), 1702 ν(IrH), 1609, 1477, 1441, 1393, 1373, 1310, 1259, 1184, 1100, 1060, 844 ν (PF₆), 775, 760, 697, 626, 560, 525, 498. ESI-MS: m/z calcd for [C₆₀H₉₃AuIr₃N₃P₄]²⁺876.7, found 876.8. Anal. calcd for C₆₀H₉₃AuF₁₂Ir₃N₃P₆: C, 35.26; H, 4.59; N, 2.06. Found: C, 35.18; H, 4.38; N, 2.04.

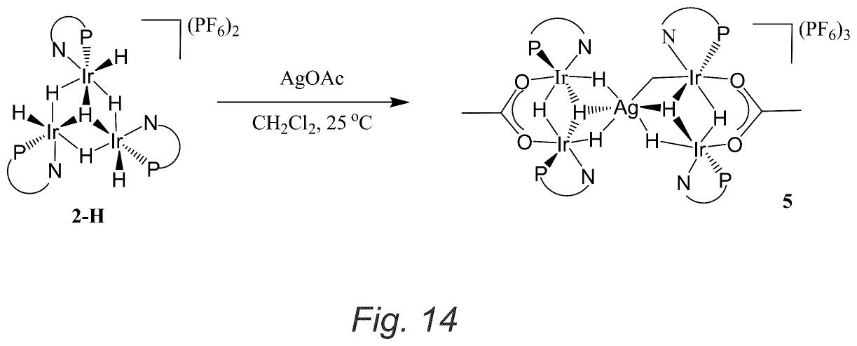

### 1.4.1.8 Compound [5](PF₆)₃ (FIG. 14)

4[Ir₃H₇(PN)₃]²⁺+11AgOAc=3[Ir₄AgH₈(OAc)₂(PN)₄]³⁺+8Ag+4AcOH+AcO⁻

A suspension of [2-H](PF₆)₂ (0.020 g, 1.261×10⁻⁵ mol) and silver (I) acetate (0.017 g, 1.009×10⁻⁴ mol, 8 eq.) in CH₂Cl₂ (1 mL) was stirred at 25° C. for two days. The resulting bright-orange solution was filtered and evaporated to dryness. Then, the residue was triturated with THF (0.25 mL) to cause crystallization of [5](PF₆)₃. The orange solid was separated by decantation, washed with THF, and dried in vacuum. Yield: 11 mg (50%). Crystals suitable for X-ray analysis were obtained by slow evaporation of CH₂Cl₂/THF solution of [5](PF₆)₃.

¹H NMR (600 MHZ, CD₂Cl₂): δ 8.59 (d, J=5.8 Hz, 2H, ArH), 7.65 (t, J=7.6 Hz, 2H, ArH), 7.60 (d, J=7.6 Hz, 2H, ArH), 7.02 (t, J=6.5 Hz, 2H, ArH), 3.64-3.48 (m, 4H, 2CH₂), 2.33 (s, 3H, CH₃), 1.18 (d, J=14.4 Hz, 18H, 6CH₃), 1.05 (d, J=13.5 Hz, 18H, 6CH₃), −4.75 (q, J=62.8 Hz, 1H, IrH), −21.85 (dd, J=29.1, 12.8 Hz, 2H, IrH), −30.78 (s, 1H, IrH). ¹³C {¹H} NMR (151 MHZ, CD₂Cl₂): δ 183.75, 163.71, 156.37, 139.69, 124.91, 122.98, 38.18 (d, ¹J_{CP}=21.1 Hz), 37.47 (d, ¹J_{CP}=28.5 Hz), 35.29 (d, ¹J_{CP}=32.4 Hz), 30.18, 28.25, 21.27. 19F {¹H} NMR (564 MHz, CD₂Cl₂): δ-72.00 (d, ¹J_{PF}=712.2 Hz, PF₆). ³¹P {¹H} NMR (243 MHZ, CD₂Cl₂): δ 66.38 (s, PN), −145.00 (hept, ¹J_{PF}=712.1 Hz, PF₆). IR (KBr, cm⁻¹): 2975, 2912, 2879, 2147 ν(IrH), 1612, 1555, 1482, 1443, 1409, 1398, 1375, 1186, 939, 848 ν (PF₆), 774, 558. MALDI-MS: m/z calcd for [C₃₀H₅₅Ir₂N₂O₂P₂]⁺923.30, found 923.16. Anal. calcd. for C60H₁₁₀AgF₁₈Ir₄N₄O₄P₇: C, 30.19; H, 4.64; N, 2.35. Found C, 30.30; H, 4.44; N, 1.78.

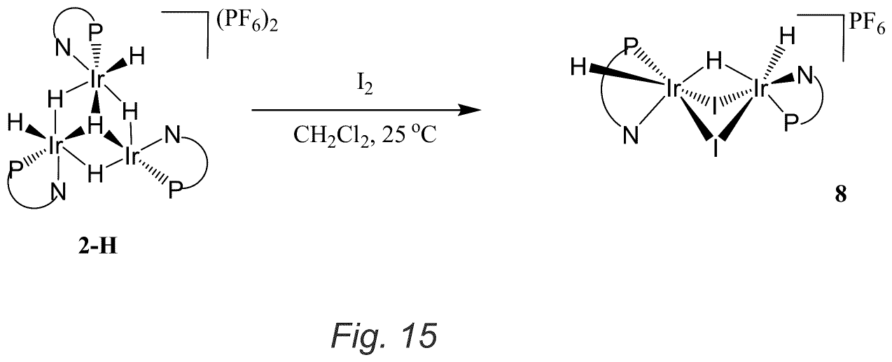

### 1.4.1.9 Compound [8] PF₆ (FIG. 15)

2[Ir₃H₇(PN)₃]²⁺+3I₂=3[Ir₂H₃I₂(PN)₂]⁺+2H₂+H⁺

A solution of iodine (0.02 M in CH₂Cl₂, 0.95 mL, 1.892× 10 5 mol, 1.5 eq.) was added drop-wise to a stirred solution of [2-H](PF₆)₂ (0.020 g, 1.261× 10 5 mol) in CH₂Cl₂ (1 mL) at room temperature. After five-hour stirring the initially orange solution turned yellow, and then the solvent was evaporated in vacuum to dryness. The residue was redissolved in CH₂Cl₂ (0.5 mL) and the product was crystallized over the course of two days by adding ether in small portions. Bright yellow cotton-like crystals of [8] PF₆ were separated by decantation, washed with ether, and dried in vacuum. Yield: 18.4 mg (77%). Crystals suitable for X-ray analysis were obtained by slow evaporation of CH₂Cl₂/hexane solution of [8] PF₆.

¹H NMR (600 MHZ, CD₂Cl₂): δ 10.07 (d, J=5.8 Hz, 1H, ArH), 9.30 (d, J=6.0 Hz, 1H, ArH), 7.89 (t, J=7.6 Hz, 1H, ArH), 7.84 (t, J=7.7 Hz, 1H, ArH), 7.75 (d, J=7.8 Hz, 1H, ArH), 7.65 (d, J=7.9 Hz, 1H, ArH), 7.12 (t, J=6.6 Hz, 1H, ArH), 7.05 (t, J=6.7 Hz, 1H, ArH), 3.92-3.79 (m, 2H, CH₂), 3.57 (dd, J=17.0, 9.4 Hz, 1H, CH₂), 3.29 (dd, J=17.2, 9.3 Hz, 1H, CH₂), 1.50 (d, J=14.8 Hz, 18H, 6CH₃), 1.39 (d, J=14.4 Hz, 9H, 3CH₃), 1.21 (d, J=14.3 Hz, 9H, 3CH₃), −18.72 (dd, ²J_{PH}=48.3, 9.0 Hz, 1H, μ-IrH), −23.30 (d, ²J_{PH}=16.3 Hz, 1H, IrH), −24.99 (d, ²J_{PH}=18.9 Hz, 1H, IrH). ¹³C {¹H} NMR (151 MHZ, CD₂Cl₂): δ 165.97, 164.44, 159.98, 158.74, 138.91, 138.89, 124.92, 124.28, 123.88 (d, ²J_{CP}=8.9 Hz), 123.41 (d, ²J_{CP}=9.1 Hz), 38.91 (d, ¹J_{CP}=29.4 Hz), 38.42 (d, ¹J_{CP}=29.7 Hz), 37.63 (d, ¹J_{CP}=19.6 Hz), 36.08 (d, ¹J_{CP}=18.2 Hz), 35.86 (d, ¹J_{CP}=20.0 Hz), 35.84 (d, ¹J_{CP}=20.5 Hz) 30.61 (d, ²J_{CP}=2.3 Hz), 30.18, 28.59, 28.42 (d, ²J_{CP}=2.8 Hz). ¹⁹F {¹H} NMR (564 MHz, CD₂Cl₂): δ-73.26 (d, ¹J_{PF}=710.7 Hz, PF₆). ³¹P {¹H} NMR (243 MHz, CD₂Cl₂): δ 70.32 (dd, J=46.2, 6.4 Hz, PN), 68.84 (t, J=9.0 Hz, PN), −145.00 (hept, ¹J_{PF}=710.7 Hz, PF₆). IR (KBr, cm⁻¹): 2954, 2907, 2876, 2243 ν(IrH), 1738 ν(IrH), 1609, 1478, 1448, 1394, 1373, 1182, 847 ν (PF₆), 773, 560. MALDI-MS: m/z calcd for [C28H₅₁I₂Ir₂N₂P₂]⁺1115.08, found 1115.03. Anal. calcd. for C28H₅₁F₆I₂Ir₂N₂P₃: C, 26.67; H, 4.08; N, 2.22. Found C, 27.05; H, 4.34; N, 2.27.

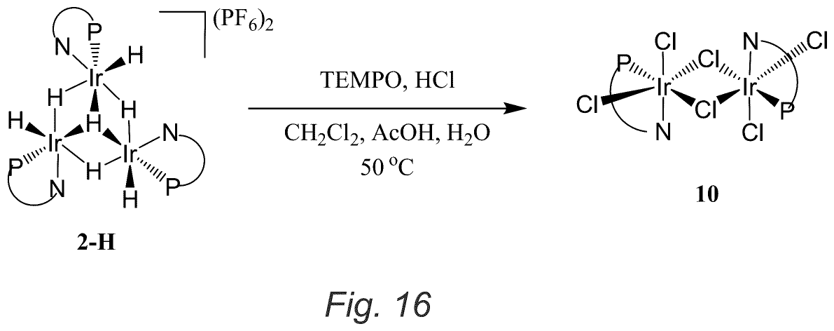

### 1.4.1.10 Compound [10]×CH₂Cl₂ (FIG. 16)

2[Ir₃H₇(PN)₃]²⁺+28TEMPO+42H⁺+18Cl⁻=3[Ir₂Cl₆(PN)₂]+28[TEMPOH₂]⁺

A mixture of [2-H](PF₆)₂ (20 mg, 1.265×10⁻⁵ mol), TEMPO (28 mg, 1.771×10⁻⁴ mol, 14 eq.), HCl (12 M in H₂O, 0.05 mL, 6×10⁻⁴ mol), acetic acid (0.5 mL), and CH₂Cl₂ (2 mL) was heated at 50° C. for 17 hours in a closed vial without stirring. During the reaction red crystals of [10]×CH₂Cl₂ were formed. Pale-yellow solution was decanted, the crystals were washed with acetone three times and then dried in vacuum. Yield: 15 mg (68%).

The obtained sample contains crystals suitable for the single-crystal X-ray analysis, which established the composition of [10]×CH₂Cl₂. The data of elemental analysis, however, slightly deviate from that composition. The compound is air-stable and insoluble in any common solvent, which precluded its characterization by NMR spectroscopy.

IR (KBr, cm⁻¹): 2942, 2907, 2876, 1612, 1477, 1451, 1393, 1369, 1316, 1268, 1184, 1163, 939, 834, 759, 742. MALDI-MS: m/z calcd for [C₂₈H₄₈Cl₅Ir₂N₂P₂]⁺1035.09, found 1035.22. Anal. calcd. for C29H₅₀Cl₈Ir₂N₂P₂: C, 30.11; H, 4.36; N, 2.42. Found C, 28.51; H, 4.00; N, 2.36.

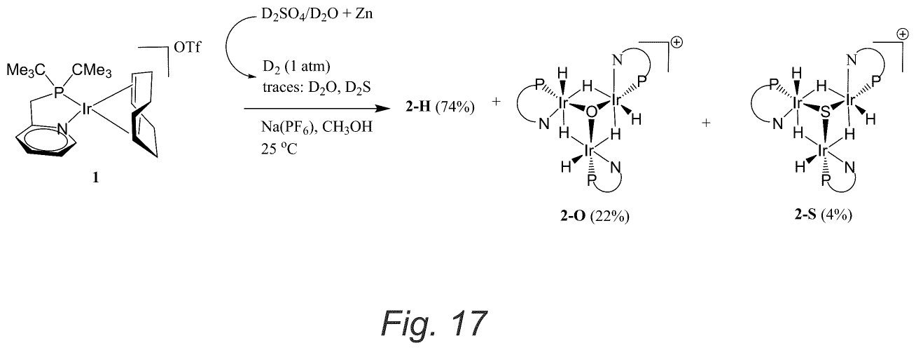

### 1.4.1.11 Generation of Complexes 2-H, 2-O, and 2-S(FIG. 17)

The experiment was performed in a fume hood. Deuterium gas contaminated with D₂O and D₂S was generated in a 50 mL Schlenk flask. The flask was charged with arbitrary amount of zinc granules, copper (II) acetate (to speed up the reaction), and D₂O (10 mL). Gas generation was initiated by injecting 98% D₂SO₄ (2 mL) through the rubber septum. Another Schlenk flask was charged with [1] OTf (0.100 g, 1.456×10⁻⁴ mol), NaPF₆ (0.050 g, 2.977×10⁻⁴ mol), methanol (5 mL), and a stir bar under N₂ atmosphere of a glovebox. The solution was stirred under the stream of D₂ for 45 min and then transferred to the glovebox. The resulting solution was evaporated to dryness, redissolved in CD₂Cl₂ (0.7 mL), and analyzed by NMR.

### 1.4.1.12 Characterization of 2-S in CD₂Cl₂ Solution

¹H NMR (400 MHZ, CD₂Cl₂): δ-22.63 (d, ²J_{PH}=22.6 Hz, 3H, IrH), −24.65 (m, 3H, IrH). ³¹P {¹H} NMR (202 MHZ, CD₂Cl₂): δ 67.29 (d, ²J_{PH}=22.3 Hz). MALDI-MS: m/z calcd for [C₄₂H₇₈Ir₃N₃P₃S]⁺1326.40, found 1326.65.

### 1.4.1.13 Hydrogenation of [1] OTf in CD₂Cl₂

2[Ir(C₈H₁₂)(PN)]⁺+6H₂+2CD₂Cl₂=[Ir₂H₄(CD₂Cl₂)₂(PN)₂]²⁺+2C₈H₁₆

A solution of [1] OTf (5 mg, 7.278×10⁻⁶ mol) in CD₂Cl₂ (0.5 mL) was treated with H₂ gas (1 atm) inside J. Young NMR tube and the hydrogenation reaction was monitored by ¹H and ³¹P NMR spectroscopy over the course of three days. The NMR data were used to identify the products and to derive the reaction kinetic profile (FIG. 5). The structure of anti-3 was established by correlating its hydride signals (Table 4) with those of crystallographically characterized analog anti-[Ir₂H₄ (CH₂Cl₂)₂

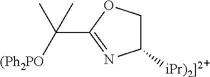

(δ_{H}=−16.14 and −26.45 ppm). ^{[2a]} The structure of syn-4 was assigned by correlating its hydride signals with previously reported complex syn-[Ir₂H₄ (CD₂Cl₂)₂

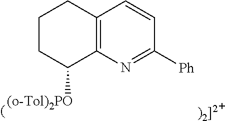

(δ_{H}=−3.32, −26.98, and −30.93 ppm). ^{[2b]}

TABLE 4
Characteristic NMR signals of complexes
anti-3 and syn-4 (CD₂Cl₂, 25° C.).

| Complex | δ(¹H), ppm | δ(³¹P), ppm |
|---|---|---|
| anti-3 | −16.91 (dd,²J_{PH} = 55.3, 9.5 Hz, | 56.54 (ddd, |
| | 2H bridging) | ²J_{PH} = 49.2, |
| | −29.55 (d, ²J_{PH} = 18.5 Hz, | 14.8, 8.7 Hz) |
| | 2H terminal) | 62.51 (s) |
| syn-4 | −3.55 (t, ²J_{trans-PH} = 73.7 Hz, | |
| | 1H bridging) | |
| | −28.32 (tt, J = 7.9, 4.2 Hz, | |
| | 1H bridging) | |
| | −29.69 (dm, J = 13.4 Hz, | |
| | 2H terminal) | |

### 1.4.1.14 Reaction Between [2-H](OTf)₂ and CF₃SO₃D

A solution of [2-H](OTf)₂ (0.010 g, 6.274×10⁻⁶ mol) in neat CF₃SO₃D (0.7 mL) was transferred to J. Young tube and then analyzed by ¹H NMR. After 3 days at 25° C. a partial deuteration of the hydride ligands was observed.

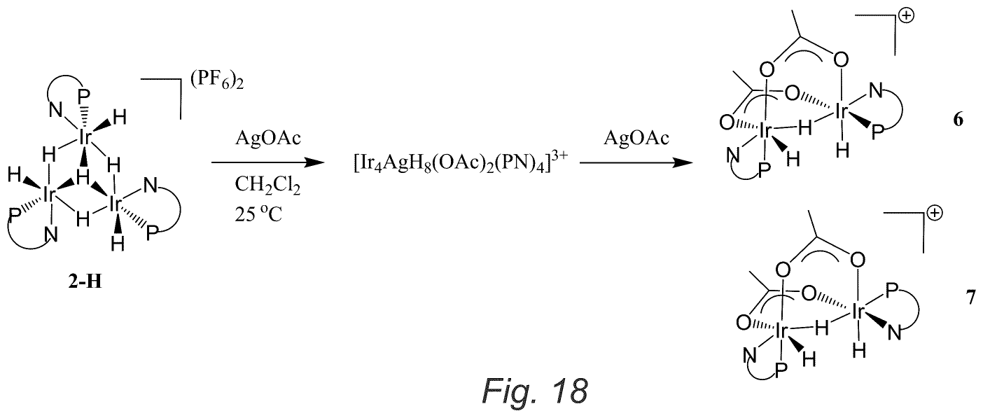

### Reaction Between [2-H](PF₆)₂ and AgOAc (FIG. 18)

4[Ir₃H₇(PN)₃]²⁺+11AgOAc=3[Ir₄AgH₈(OAc)₂(PN)₄]³⁺+8Ag+4AcOH+AcO⁻

[Ir₄AgH₈(OAc)₂(PN)₄]³⁺+3AgOAc=2[Ir₂H₃(OAc)₂(PN)₂]⁺+4Ag+AcOH+H⁺

2[Ir₃H₇(PN)₃]²⁺+10AgOAc=3[Ir₂H₃(OAc)₂(PN)₂]⁺+10Ag+4AcOH+H⁺

Characteristic hydride signals of complexes 6 and 7: ¹H NMR (600 MHZ, CD₂Cl₂): δ-19.57 (dd, J=50.7, 14.2 Hz, 1H, 7), −24.51 (d, J=16.1 Hz, 1H, 7), −26.21 (d, J=20.9 Hz, 1H, 7), −26.30 (dd, J=19.0, 2.8 Hz, 2H, 6), −28.60 (t, J=9.5 Hz, 1H, 6).

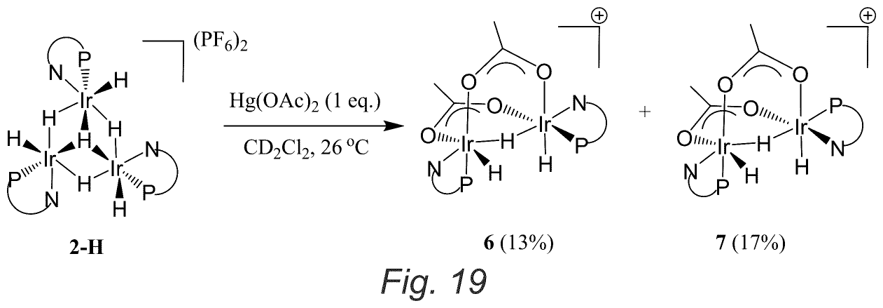

### 1.4.1.15 Reaction Between [2-H](PF₆)₂ and Hg(OAc)₂ (FIG. 19)

2[Ir₃H₇(PN)₃]²⁺+5Hg(OAc)₂=3[Ir₂H₃(OAc)₂(PN)₂]⁺+5Hg+4HOAc+H⁺

Compound [2-H](PF₆)₂ (20 mg, 1.261× 10-5 mol) and Hg(OAc)₂ (4 mg, 1.261×10⁻⁵ mol) were mixed with CD₂Cl₂ (0.5 mL) and stirred at room temperature until Hg(OAc)₂ completely dissolved. During the reaction metallic mercury precipitated and the solution turned orange. After 6 hours the solution was analyzed by ¹H NMR, which revealed partial conversion of 2-H to a complex mixture of intermediates containing Ir—H bonds. After 3 days the solution turned bright yellow and previously observed intermediates converged to isomeric complexes 6 (13%) and 7 (17%). Combined yield of 6 and 7 was used to determine the reaction stoichiometry.

### 1.4.1.16 Reaction Between [8] PF₆ and AgOAc

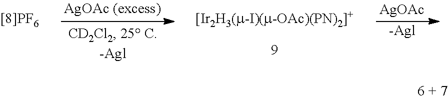

### 1.4.1.17 Characteristic Hydride Signals of Complex 9

¹H NMR (600 MHZ, CD₂Cl₂): δ-18.30 (dd, ²J_{trans-PH}=50.5, 10.4 Hz, 1H), −21.90 (d, J=20.2 Hz, 1H), −27.15 (d, J=18.7 Hz, 1H).

### 1.4.1.18 Room-Temperature Hydrogenation of CO₂

A solution of [2-H](PF₆)₂ (10 mg, 6.307× 10-6 mol), K₂CO₃ (7 mg, 5.065× 10-5 mol), and DI water (5 L) in CD₃OD (0.5 mL) was transferred to J. Young tube, and then the head space was charged with 1 bar of H₂/CO₂ (1:1). The reaction was conducted at room temperature and monitored by ¹H NMR.

### 1.4.1.19 Catalytic Dehydrogenation of HCOOH

Compound [2-H](PF₆)₂ (8.5 mg, 5.361× 10-6 mol), HCOONa (1.20 g, 18 mmol), and HCOOH (3 mL) were placed in a round-bottom flask equipped with a reflux condenser and a gas outlet. The solution was heated at 110° C. and once fully dehydrogenated the mixture was recharged with HCOOH (10 times by 3 mL). In total, 33 mL of HCOOH were dehydrogenated over the course of 4 days (TON=54400 per Ir atom).

### 1.4.1.20 Cyclic Voltammetry Experiments

Cyclic voltammetry experiments were performed in a single compartment electrochemical cell under nitrogen atmosphere using glassy carbon electrode (surface area of 0.195 cm²) as the working electrode, platinum wire as the auxiliary electrode, and silver wire as the reference electrode. The working solution was prepared under nitrogen atmosphere in a glovebox by mixing [2-H](PF₆)₂ (16 mg, 1×10⁻⁵ mol, 0.33 mM), tetrabutylammonium hexafluorophosphate (0.387 g, 1×10⁻³ mol, 0.033 M), dichloromethane (10 mL), methanol (10 mL), and DI water (10 mL). The resulting homogeneous solution was transferred to the electrochemical cell via syringe and cyclic voltammograms were recorded within 2.0 to −2.3 V potential window at 50 and 200 mV/s scan rates. The potentials were referenced to the redox couple of decamethylferrocene internal standard. The voltammograms reveal four chemically irreversible redox events: two oxidations at 0.73 and 1.74 V and two reductions at −1.67 and −0.90 V (at 200 mV/s). Increasing the scan rate above 200 mV/s does not bring about new redox features.

### 1.4.1.21 Crystallographic Studies

X-Ray Crystallography. Crystals were mounted on a Rigaku XtaLAB Synergy, Dualflex, Hypix diffractometer from microfocus sealed tube for X-ray crystallographic collection with Mo Kα radiation source (2=0.71073 Å). The crystals were kept at 100-160 K during data collection. The structure was solved by intrinsic phasing using SHELXTL XT 2014/5 (Bruker AXS, 2014) structure solution program and refined on SHELXTL XL 2018/3 (Bruker AXS, 2018) refinement package using full-matrix least squares minimization on F^{2.[10-13]} All non-hydrogen atoms were refined anisotropically. Crystal data and experimental parameters for all structures are given in Table 5. Further crystallographic details can be obtained from the Cambridge Crystallographic Data Centre.

TABLE 5
Crystal data and experimental parameters for compounds [2-H](OTf)₂ × CH₂Cl₂,
[2-O]OTf, [2-Au](BF₄)(PF₆), [5](PF₆)₃ × ¼CH₂Cl₂, and [10] × CH₂Cl₂.

| Compound | [2-H](OTf)₂ × CH₂Cl₂ | | [2-O]OTf | | [2-Au](BF₄)(PF₆) | |
|---|---|---|---|---|---|---|
| CCDC | 2289546 | | 2289544 | | 2289547 | |
| Chemical formula | [C₄₂H₇₉Ir₃N₃P₃] | | [C₄₂H₇₈Ir₃N₃OP₃] | | [C₆₀H₈₇AuIr₃N₃P₄] | |
| | [CF₃SO₃]_{2 ×} CH₂Cl₂ | | [CF₃SO₃] | | [BF₄][PF₆] | |
| Formula weight (g/mol) | 1678.65 | | 1459.65 | | 1979.55 | |
| Crystal color | yellow | | yellow | | red | |
| Crystal size (mm³) | 0.35 × 0.14 × 0.07 | | 0.15 × 0.13 × 0.10 | | 0.31 × 0.16 × 0.06 | |
| Crystal system | orthorhombic | | trigonal | | monoclinic | |
| Space group | Pbca (no. 61) | | P-3 (no. 147) | | P2₁/c (no. 14) | |
| Unit cell dimensions | | | | | | |
| a (Å) | 19.1876 | (2) | 13.9561 | (2) | 13.1450 | (2) |
| b (Å) | 20.3309 | (2) | 13.9561 | (2) | 28.6637 | (3) |
| c (Å) | 31.4163 | (3) | 17.1572 | (2) | 19.6662 | (2) |
| α (°) | 90 | | 90 | | 90 | |
| β (°) | 90 | | 90 | | 97.4670 | (10) |
| γ (°) | 90 | | 120 | | 90 | |
| Volume (Å³) | 12255.5 | (2) | 2894.04 | (9) | 7347.08 | (16) |

| Z | 8 | 2 | | 4 | |
|---|---|---|---|---|---|
| D_{calc} (g/cm³) | 1.820 | 1.675 | | 1.790 | |
| Temperature (K.) | 160.15 | 100.01 | (10) | 99.98 | (13) |

| | | | |
|---|---|---|---|
| Abs. coefficient (mm⁻¹) | 6.794 | 7.045 | 7.577 |
| F(000) | 6528.0 | 1416 | 3792 |
| 2θ range (°) | 4.694 to 66.54 | 4.748 to 59.15 | 4.748 to 68.042 |
| Reflections collected | 351608 | 74630 | 237485 |
| Independent reflections | 22209 | 5428 | 27065 |
| Data/restraints/parameters | 22209/0/685 | 5428/0/195 | 27065/12/766 |
| R-factor (%) | 2.52 | 1.97 | 4.47 |
| Goodness-of-fit on F² | 1.035 | 1.047 | 1.044 |
| Δ/σₘₐₓ | 0.010 | 0.001 | 0.004 |
| Weighting scheme | w = 1/[σ²(Fₒ²) + | w = 1/[σ²(Fₒ²) + | w = 1/[σ²(Fₒ²)+ |
| | (0.0101P)² + 35.8022P], | (0.0198P)² + 4.6719P], | (0.0146P)² + 99.9299P], |
| | P = (Fₒ² + 2F_{c}²)/3 | P = (Fₒ² + 2F_{c}²)/3 | P = (Fₒ² + 2F_{c}²)/3 |
| Largest diff. peak/hole | 1.59/−1.02 eÅ⁻³ | 1.61 /−0.79 eÅ⁻³ | 3.79/−2.79 eÅ⁻³ |

| Compound | [5](PF₆)₃ × ¼CH₂Cl₂ | | [10] × CH₂Cl₂ | |
|---|---|---|---|---|
| CCDC | 2289545 | | 2289543 | |
| Chemical formula | [C₆₀H₁₀₄AgIr₄N₄O₄P₄] | | [C₂₈H₄₈Cl₆Ir₂N₂P₂] × CH₂Cl₂ | |
| | [PF₆]₃ × ¼CH₂Cl₂ | | | |
| Formula weight (g/mol) | 2402.15 | | 1156.65 | |
| Crystal color | orange | | red | |
| Crystal size (mm³) | 0.183 × 0.095 × 0.056 | | 0.16 × 0.10 × 0.05 | |
| Crystal system | triclinic | | monoclinic | |
| Space group | P-1 (no. 2) | | I2/a (no. 15) | |
| Unit cell dimensions | | | | |
| a (Å) | 15.5554 | (2) | 14.9806 | (6) |
| b (Å) | 16.2715 | (3) | 14.9616 | (4) |
| c (Å) | 19.3146 | (3) | 17.5808 | (7) |
| α (°) | 99.4470 | (10) | 90 | |
| β (°) | 101.9520 | (10) | 108.934 | (4) |
| γ (°) | 111.917 | (2) | 90 | |
| Volume (Å³) | 4277.19 | (13) | 3727.2 | (2) |
| Z | 2 | | 4 | |
| D_{calc} (g/cm³) | 1.865 | | 2.061 | |
| Temperature (K.) | 100.00 | (10) | 100 | (2) |

| | | |
|---|---|---|
| Abs. coefficient (mm⁻¹) | 6.652 | 7.818 |
| F(000) | 2313.0 | 2232 |
| 2θ range (°) | 4.02 to 65.798 | 2.723 to 30.996 |
| Reflections collected | 116384 | 18132 |
| Independent reflections | 26670 | 4792 |
| Data/restraints/parameters | 26670/0/980 | 4792/0/201 |
| R-factor (%) | 3.36 | 5.10 |
| Goodness-of-fit on F² | 1.043 | 0.971 |
| Δ/σₘₐₓ | 0.002 | 0.001 |
| Weighting scheme | w = 1/[σ²(Fₒ²) + (0.0395P)² + | w = 1/[σ²(Fₒ²) + (0.0762P)²], |
| | 4.6090P], | P = (Fₒ² + 2F_{c}²)/3 |
| | P = (Fₒ² + 2F_{c}²)/3 | |
| Largest diff. peak/hole | 2.11/−1.40 eÅ⁻³ | 1.923/−2.21 eÅ⁻³ |

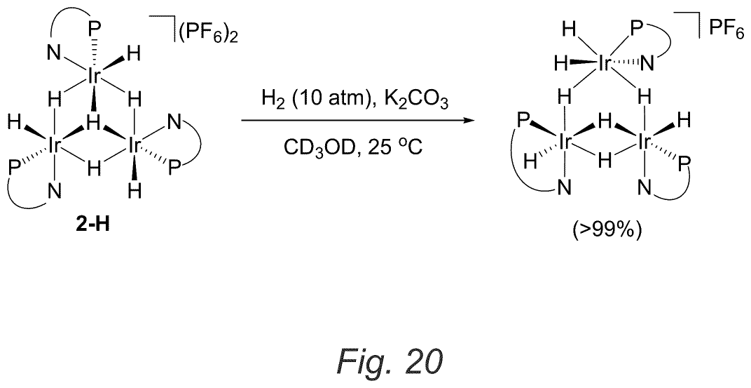

### 2. Preparation of Compound VII (FIG. 20)

While it is inert to CO₂ at various pressures, treating compound 2-H (I) with hydrogen gas in the presence of a base, K₂CO₃, results in opening the cluster at its 13 hydride with uptake of H₂. This results in the installation of an eighth hydride in the cluster with concurrent reduction in charge from +2 to +1. This appears to proceed through an initial coordinated H₂ structure that is deprotonated to form the product. Uptake of H₂ by the cluster appears to be reversible, whereas an elevated pressure of H₂, 10 atm, is required to enable an overall yield of >99%.

### 3. Mechanistic Study of Complexes IVA and IVb

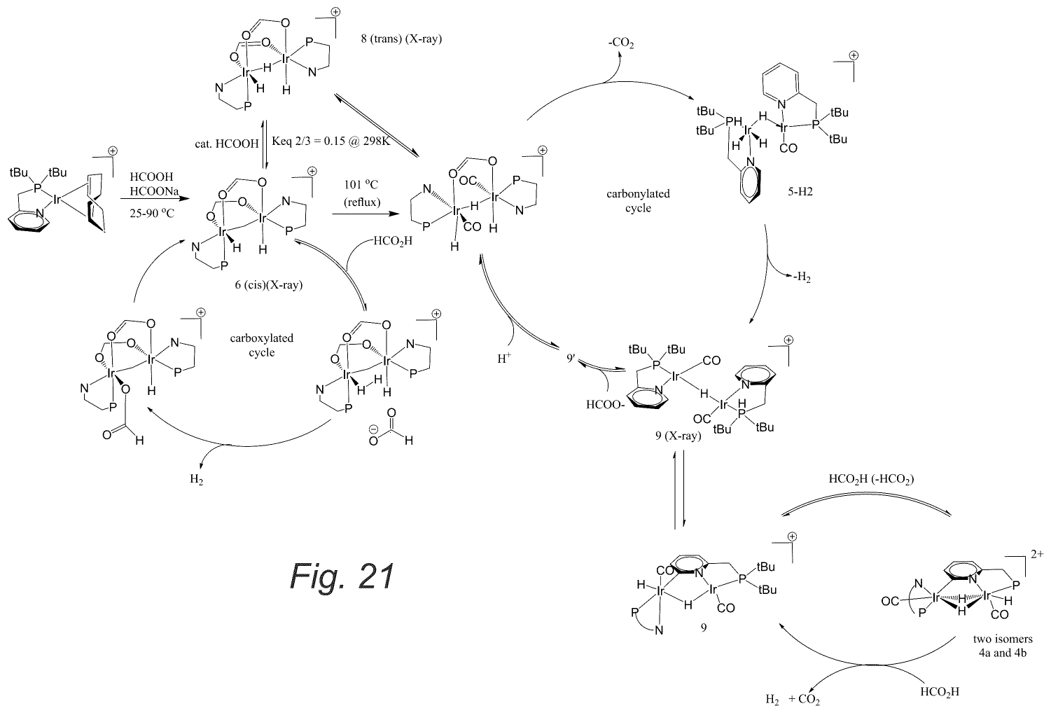

FIG. 21 depicts a mechanistic study show the formation of organometallic complexes 16 and IVa and IVb.

While exemplary embodiments are described above, it is not intended that these embodiments describe all possible forms of the invention. Rather, the words used in the specification are words of description rather than limitation, and it is understood that various changes may be made without departing from the spirit and scope of the invention. Additionally, the features of various implementing embodiments may be combined to form further embodiments of the invention.

### REFERENCES

1. a) D. Baidilov, D. Hayrapetyan, A. Y. Khalimon, Tetrahedron 2021, 98, 132435; b) R. H. Crabtree, ACS Sustain. Chem. Eng. 2019, 7, 15845; c) G. E. Dobereiner, R. H. Crabtree, Chem. Rev. 2010, 110, 681; d) V. Cherepakhin, T. J. Williams, ACS Catal. 2018, 8, 3754.
2. a) J. R. Cabrero-Antonino, R. Adam, V. Papa, M. Beller, Nat. Commun. 2020, 11, 3893; b) A. Nalikezhathu, V. Cherepakhin, T. J. Williams, Org. Lett. 2020, 22, 4979; c) A. Nalikezhathu, A. Tam, V. Cherepakhin, V. K. Do, T. J. Williams, Org. Lett. 2023, 25, 1754; d) R. Qu, Y. Cheng, S. Yang, C. Zhao, H. Liu, X. Huang, ChemistrySelect 2021, 6, 4089; e) Z. Yin, H. Zeng, J. Wu, S. Zheng, G. Zhang, ACS Catal. 2016, 6, 6546; f) V. Cherepakhin, T. J. Williams, ACS Catal. 2020, 10, 56.
3. a) K. Manickam, P. Mistry, G. Walker, D. Grant, C. E. Buckley, T. D. Humphries, M. Paskevicius, T. Jensen, R. Albert, K. Peinecke, M. Felderhoff, Int. J. Hydrog. 2019, 44, 7738; b) A. Schneemann, J. L. White, S. Kang, S. Jeong, L. F. Wan, E. S. Cho, T. W. Heo, D. Prendergast, J. J. Urban, B. C. Wood, M. D. Allendorf, V. Stavila, Chem. Rev. 2018, 118, 10775.
4. R. Kanega, N. Onishi, S. Tanaka, H. Kishimoto, Y. Himeda, J. Am. Chem. Soc. 2021, 143, 1570.
5. S. Hu, T. Shima, Z. Hou, Nature 2014, 512, 413.
6. a) D. Singh, W. R. Buratto, J. F. Torres, L. J. Murray, Chem. Rev. 2020, 120, 5517; b) S. F. McWilliams, P. L. Holland, Acc. Chem. Res. 2015, 48, 2059; c) Y. Lee, F. T. Sloane, G. Blondin, K. A. Abboud, R. García-Serres, L. J. Murray, Angew. Chem., Int. Ed. 2015, 54, 1499.
7. a) A. Brenner, D. A. Hucul, J. Am. Chem. Soc. 1980, 102, 2484; b) A. C. Ghosh, C. Duboc, M. Gennari, Coord. Chem. Rev. 2021, 428, 213606; c) D. Mahajan, Top. Catal. 2005, 32, 209.
8. a) S. Moret, P. J. Dyson, G. Laurenczy, Nat. Commun. 2014, 5, 4017; b) R. Tanaka, M. Yamashita, K. Nozaki, J. Am. Chem. Soc. 2009, 131, 14168; c) W. H. Wang, J. F. Hull, J. T. Muckerman, E. Fujita, Y. Himeda, Energy Environ. Sci. 2012, 5, 7923; d) N. Westhues, M. Belleflamme, J. Klankermayer, ChemCatChem 2019, 11, 5269; e) A. J. M. Miller, J. A. Labinger, J. E. Bercaw, Organometallics 2011, 30, 4308.
9. a) M. Hanifa, R. Agarwal, U. Sharma, P. C. Thapliyal, L. P. Singh, J. CO₂ Util. 2023, 67, 102292; b) F. N. U. Huhe, J. King, S. S. C. Chuang, Res. Chem. Intermed. 2023, 49, 791; c) C. J. Koch, V. Galvan, A. Goeppert, G. K. S. Prakash, Green Chem. 2023, 25, 1803; d) B. Shao, Z. Q. Wang, X. Q. Gong, H. Liu, F. Qian, P. Hu, J. Hu, Nat. Commun. 2023, 14, 996.
10. Z. Ma, U. Legrand, E. Pahija, J. R. Tavares, D. C. Boffito, Ind. Eng. Chem. Res. 2021, 60, 803.
11. S. C. Ricke, D. K. Dittoe, K. E. Richardson, Front. Vet. Sci. 2020, 7:563.
12. H. A. Evans, D. Mullangi, Z. Deng, Y. Wang, S. B. Peh, F. Wei, J. Wang, C. M. Brown, D. Zhao, P. Canepa, A. K. Cheetham, Sci. Adv. 2022, 8, eade 1473.
13. a) D. F. Chodosh, R. H. Crabtree, H. Felkin, G. E. Morris, J. Organomet. Chem. 1978, 161, C67; b) D. F. Chodosh, R. H. Crabtree, H. Felkin, S. Morehouse, G. E. Morris, Inorg. Chem. 1982, 21, 1307.
14. a) H. H. Wang, L. H. Pignolet, Inorg. Chem. 1980, 19, 1470; b) H. H. Wang, A. L. Casalnuovo, B. J. Johnson, A. M. Mueting, L. H. Pignolet, Inorg. Chem. 1988, 27, 325.
15. S. P. Smidt, A. Pfaltz, E. Martínez-Viviente, P. S. Pregosin, A. Albinati, Organometallics 2003, 22, 1000.
16. S. shitaya, K. Nomura, A. Inagaki, Dalton Trans. 2018, 47, 12046.
17. a) S. Gruber, M. Neuburger, A. Pfaltz, Organometallics 2013, 32, 4702; b) M. A. Muller, S. Gruber, A. Pfaltz, Adv. Synth. Catal. 2018, 360, 1340.
18. M. R. Churchill, J. P. Hutchinson, Inorg. Chem. 1978, 17, 3528.
19 V. F. Sears, Neutron News 1992, 3, 26.
20 Jmol: an open-source Java viewer for chemical structures in 3D. http://www.jmol.org/
21. a) R. H. Morris, Inorg. Chem. 2018, 57, 13809; b) M. A. Esteruelas, M. Oliván, A. Vélez, Inorg. Chem. 2013, 52, 12108; c) J. C. Lee, A. L. Rheingold, B. Muller, P. S. Pregosin, R. H. Crabtree, J. Chem. Soc., Chem. Commun. 1994, 1021; d) S. A. Smith, D. M. Blake, M. Kubota, Inorg. Chem. 1972, 11, 660.
22. R. D. Pergola, F. Demartin, L. Garlaschelli, M. Manassero, S. Martinengo, N. Masciocchi, M. Sansoni, Organometallics 1991, 10, 2239.
23. T. Chihara, M. Sato, H. Konomoto, S. Kamiguchi, H. Ogawa, Y. Wakatsuki, J. Chem. Soc., Dalton Trans. 2000, 2295.
24. A. L. Casalnuovo, L. H. Pignolet, J. W. A. van der Velden, J. J. Bour, J. J. Steggerda, J. Am. Chem. Soc. 1983, 105, 5957.
25. S. G. Bratsch, J. Phys. Chem. Ref. Data 1989, 18, 1.
26. F. Bachechi, J. Organomet. Chem. 1994, 474, 191.
27. M. L. Li, S. Yang, X. C. Su, H. L. Wu, L. L. Yang, S. F. Zhu, Q. L. Zhou, J. Am. Chem. Soc. 2017, 139, 541.
28. J. J. A. Celaje, Z. Lu, E. A. Kedzie, N. J. Terrile, J. N. Lo, T. J. Williams, Nature Commun. 2016, 7:11308.
29. Deposition numbers 2289546, 2235972 (for [2-H](OTf)2×CH2Cl2), 2289544 (for [2-O] OTf), 2289547 (for [2-Au] (BF4) (PF6)), 2289545 (for [5](PF6)3×¼CH2Cl2), and 2289543 (for [10]×CH2Cl2) contain the supplementary crystallographic data for this paper. These data are provided free of charge by the joint Cambridge Crystallographic Data Centre and Fachinformationszentrum Karlsruhe Access Structures service.

What is claimed is:

1. An organometallic complex having formula I: 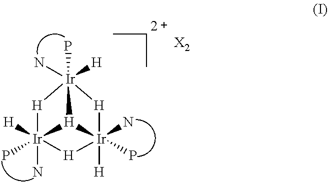
wherein:
X is a counterion having a charge of −1 or X₂ is a bivalent anion; and 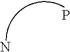
is a bidentate ligand having nitrogen and phosphorus as coordinating atoms.

2. The organometallic complex of claim 1 wherein: 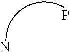
is 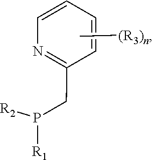
n is 0, 1, 2, 3, and 4;
R₁, R₂ are C₁₋₁₀ alkyl; and
R₃ is C₁₋₁₀ alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl and M is a metal atom.

3. The organometallic complex of claim 1 wherein R₁, R₂ are each independently methyl, ethyl, butyl, n-propyl, isopropyl, n-butyl, sec-butyl, t-butyl.

4. The organometallic complex of claim 1 wherein R₁, R₂ are t-butyl.

5. The organometallic complex of claim 1 wherein R₃ is C₁₋₁₀ alkyl.

6. A method for hydrogenation CO₂ to form a formate, the method comprising:
contacting the organometallic complex having formula 1 of claim 1 with CO₂ and H₂.

7. A method for dehydrogenation of formic acid, the method comprising:
contacting the organometallic complex having formula 1 of claim 1 with formic acid.

8. An organometallic complex having formula II: 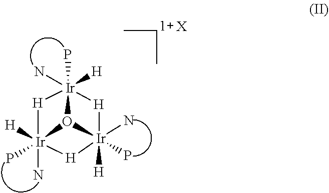
wherein:
X is a counterion having a charge of −1; and 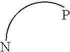
is a bidentate ligand having nitrogen and phosphorus as coordinating atoms.

9. The organometallic complex of claim 8, wherein: 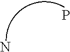
is 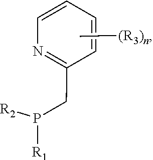
n is an integer from 0 to 4;
R₁, R₂ are C₁₋₁₀ alkyl; and
R₃ is C₁₋₁₀ alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃⁻M⁺, —COO⁻M⁺, —CF₂H, —CF₂R′, —CFH₂, and —CFR′R″ where R′, R″ and R′″ are C₁₋₁₀ alkyl and M is a metal atom.

10. The organometallic complex of claim 9, wherein R₁, R₂ are each independently methyl, ethyl, butyl, n-propyl, isopropyl, n-butyl, sec-butyl, t-butyl.

11. The organometallic complex of claim 9, wherein R₁, R₂ are t-butyl.

12. The organometallic complex of claim 9, wherein R₃ is C₁₋₁₀ alkyl.

13. A method for hydrogenation of CO₂ to form a formate, the method comprising:
contacting the organometallic complex having formula 2 of claim 8 with CO₂ and H₂.

14. A method for dehydrogenation of formic acid, the method comprising:
contacting the organometallic complex having formula II with formic acid: 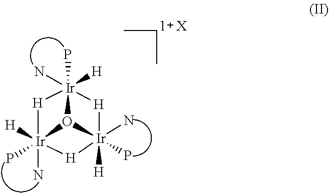
wherein:
X is a counterion having a charge of −1; and 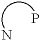
is a bidentate ligand having nitrogen and phosphorus as coordinating atoms.

15. An organometallic complex having formula III:
wherein: 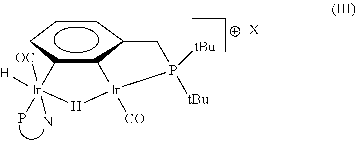
X is a counterion having a charge of −1; and 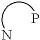
is a bidentate ligand having nitrogen and phosphorus as coordinating atoms.

16. The organometallic complex of claim 15, wherein: 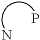
is 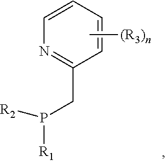
n is an integer from 0 to 4;
R₁, R₂ are C₁₋₁₀ alkyl; and
R₃ is C₁₋₁₀ alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃ M⁺, —COO⁻M⁺, —CF₂H, —CF₂R′, —CFH₂, and —CFR′R″ where R′, R″ and R′″ are C₁₋₁₀ 16 9, wherein R₁, R₂ are each independently methyl, ethyl, butyl, n-propyl, isopropyl, n-butyl, sec-butyl, t-butyl.

17. The organometallic complex of claim 16, wherein R₁, R₂ are each independently methyl, ethyl, butyl, n-propyl, isopropyl, n-butyl, sec-butyl, t-butyl.

18. The organometallic complex of claim 17, wherein R₁, R₂ are t-butyl.

19. The organometallic complex of claim 17, wherein R₃ is C₁₋₁₀ alkyl.

20. A method for hydrogenation of CO₂ to form a formate, the method comprising:
contacting the organometallic complex having formula 3 of claim 15 with CO₂ and H₂.

21. A method for dehydrogenation of formic acid, the method comprising:
contacting the organometallic complex having formula 3 of claim 15 with formic acid.

22. An organometallic complex having formula IVa or IVb: 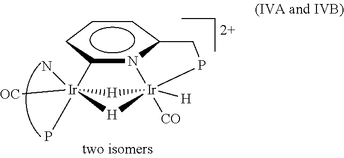
wherein:
X is a counterion having a charge of −1; and 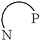
is a bidentate ligand having nitrogen and phosphorus as coordinating atoms.

23. The organometallic complex of claim 22, wherein: 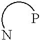
is 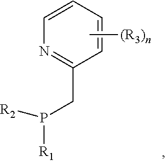
wherein:
n is an integer from 0 to 4;
R₁, R₂ are C₁₋₁₀ alkyl; and
R₃ is C₁₋₁₀ alkyl, —NO₂, —NH₂, —N(R′R″), —N(R′R″R′″)⁺L⁻, Cl, F, Br, —CF₃, —CCl₃, —CN, —SO₃H, —PO₃H₂, —COOH, —CO₂R′, —COR′, —CHO, —OH, —OR′, —O⁻M⁺, —SO₃⁻M⁺, —PO₃ M⁺, —COO⁻M⁺, —CF₂H, —CF₂R′, —CFH₂, and —CFR′R″ where R′, R″ and R′″ are C₁₋₁₀ 16 9, wherein R₁, R₂ are each independently methyl, ethyl, butyl, n-propyl, isopropyl, n-butyl, sec-butyl, t-butyl.

24. The organometallic complex of claim 23, wherein R₁, R₂ are each independently methyl, ethyl, butyl, n-propyl, isopropyl, n-butyl, sec-butyl, t-butyl.

25. The organometallic complex of claim 23, wherein R₁, R₂ are t-butyl.

26. The organometallic complex of claim 23, wherein R₃ is C₁₋₁₀ alkyl.

27. A method for hydrogenation of CO₂ to form a formate, the method comprising:
contacting the organometallic complex having formula IVA or IVB of claim 22 with CO₂ and H₂.

28. A method for dehydrogenation of formic acid, the method comprising:
contacting the organometallic complex having formula IVA or IVB with formic acid:

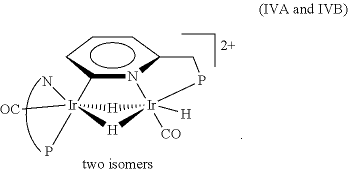

29. A method comprising reacting an organometallic complex having formula I with carbon monoxide in a solvent to form an organometallic complex having formula V: 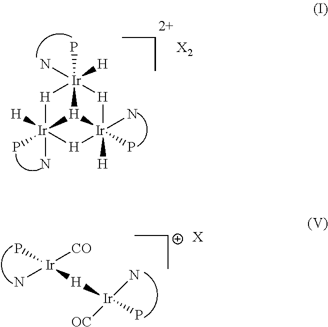
wherein:
X is a counterion having a charge of −1 or X₂ is a bivalent anion; and 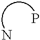
is a bidentate ligand having nitrogen and phosphorus as coordinating atoms.

30. A method comprising reacting an organometallic complex having formula I in a solvent to form an organometallic complex having formula VI: 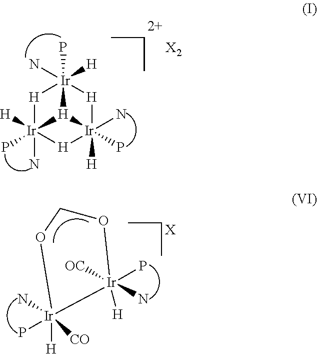
wherein:
X is a counterion having a charge of −1 or X₂ is a bivalent anion; and 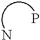
is a bidentate ligand having nitrogen and phosphorus as coordinating atoms.

31. An organometallic complex having formula VII: 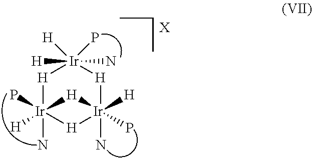
X is a counterion having a charge of −1.
